(19) United States
(12) Patent Application Publication
(10) Pub. No.: US 2025/0078960 A1
(43) Pub. Date: Mar. 6, 2025

(54) HOLOGRAPHIC QUANTUM COMPUTING WITH COMPACT QUANTUM COMPUTERS

(71) Applicant: Yale University, New Haven, CT (US)

(72) Inventors: Ningyi Lyu, New Haven, CT (US); Victor Batista, Hamden, CT (US)

(73) Assignee: Yale University, New Haven, CT (US)

(21) Appl. No.: 18/821,547

(22) Filed: Aug. 30, 2024

Related U.S. Application Data
(60) Provisional application No. 63/579,856, filed on Aug. 31, 2023.

(51) Int. Cl. G16C 20/20; G16C 20/70

(52) U.S. Cl. CPC G16C 20/20; G16C 20/70

(57) ABSTRACT

Systems and methods for implementing holographic quantum circuits on compact quantum computing systems are provided. Compact quantum computing systems include systems having fewer physical qubits than the number of quantum modes to be instantiated during execution of the quantum circuit. Techniques described herein may be used to perform boson sampling techniques, including with application to molecular docking simulations.

### CROSS-REFERENCE TO RELATED APPLICATIONS

This application claims the benefit under 35 U.S.C. § 119 (c) of U.S. Provisional Patent Application No. 63/579,856, filed Aug. 31, 2023, and titled “HOLOGRAPHIC QUANTUM COMPUTING WITH MICROWAVE CAVITY RESONATORS,” which is incorporated herein by reference in its entirety.

### GOVERNMENT FUNDING

This invention was made with government support under 2124511 awarded by the National Science Foundation. The government has certain rights in the invention.

### BACKGROUND

Quantum information processing techniques perform computation by manipulating one or more quantum objects (e.g., objects that can store quantum states and/or manipulate quantum states). These techniques are sometimes referred to as “quantum computing.” In order to perform computations, a quantum computing system utilizes quantum objects to reliably store, process, and retrieve information. According to some quantum information processing approaches, a quantum analogue to the classical computing “bit” (being equal to 1 or 0) has been developed, which is referred to as a quantum bit, or “qubit.” A qubit can be composed of any quantum system that has two distinct states (which may be thought of as 1 and 0 states), but also has the special property that the system can be placed into quantum superpositions and thereby potentially exist in both of those states at once.

The field of circuit quantum electrodynamics (cQED) represents one illustrative experimental approach to implement quantum computing. In these approaches, one or more qubit systems are each coupled to a resonator cavity in such a way as to allow mapping of the quantum information contained in the qubit(s) to and/or from the resonator(s). The resonator(s) generally will have a longer stable lifetime than the qubit(s). The quantum state may later be retrieved in a qubit by mapping the state back from a respective resonator to the qubit.

### SUMMARY

Some embodiments are directed to a method of identifying, using a compact quantum computing system, binding configurations between a first molecular structure and a second molecular structure, the method including: generating a binding interaction graph describing possible binding interactions between portions of the first molecular structure and portions of the second molecular structure, the binding interaction graph being encoded as a graph adjacency matrix; parameterizing a multimode Gaussian state vector using the graph adjacency matrix; determining binding interactions by sampling, using holographic computing implemented on the compact quantum computing system, the multimode Gaussian state vector; and identifying, using the determined binding interactions, cliques within the binding interaction graph, the cliques corresponding to stable binding configurations between the first and second molecular structures.

In some embodiments, the method further includes identifying the first molecular structure as a candidate pharmaceutical compound.

In some embodiments, the method further includes manufacturing the first molecular structure.

In some embodiments, the method further includes determining safety and/or efficacy of the first molecular structure by performing a clinical and/or nonclinical study using the first molecular structure.

In some embodiments, generating the binding interaction graph includes generating a binding interaction graph describing possible binding interactions between portions of the first molecular structure and portions of the second molecular structure, the second molecular structure including a structure describing a biological receptor or a portion of a biological receptor.

In some embodiments, using holographic computing implemented on the compact quantum computing system includes: applying a first squeezing operation to a vacuum state of a first physical qubit to generate a first quantum state in the first physical qubit; applying a second squeezing operation to a vacuum state of a second physical qubit to generate a second quantum state in the second physical qubit; causing interference between the first quantum state and the second quantum state by applying a beamsplitter interaction between the first quantum state and the second quantum state; and after applying the beamsplitter interaction: measuring the first quantum state stored in the first physical qubit; and initializing a third quantum state in the first physical qubit by applying a third squeezing operation to a vacuum state of the first physical qubit.

In some embodiments, identifying the binding interactions includes identifying a first binding interaction by detecting a photon stored in the first physical qubit by measuring the first quantum state.

In some embodiments, applying the first squeezing operation includes applying a squeezing operation having squeezing parameters determined using the graph adjacency matrix.

In some embodiments, applying the first squeezing operation to the first physical qubit includes applying an electromagnetic signal to a first ancilla qubit coupled to the first physical qubit, the electromagnetic signal being configured to cause three-wave or four-wave mixing between the first quantum state and a state of the first ancilla qubit. In some embodiments, the electromagnetic signal has a frequency approximately equal to twice a resonant frequency of the first physical qubit, the first physical qubit including a first cavity resonator.

In some embodiments, applying the beamsplitter interaction further includes determining a covariance matrix using the graph adjacency matrix; and applying a beamsplitter interaction having beamsplitting parameters based on the covariance matrix.

In some embodiments, applying the beamsplitter interaction includes applying an electromagnetic signal to a second ancilla qubit coupled between the first and second physical qubits, the electromagnetic signal having a frequency approximately equal to a difference between resonant frequencies of the first and second physical qubits, the first and second physical qubits including cavity resonators.

In some embodiments, applying the beamsplitter interaction includes applying the electromagnetic signal to a transmon qubit.

In some embodiments, applying the beamsplitter interaction includes applying the electromagnetic signal to the second ancilla qubit, the electromagnetic signal configured to cause four-wave mixing between the first quantum state and the second quantum state.

In some embodiments, the method further includes: applying a third squeezing operation to a vacuum state of a third physical qubit to generate a fourth quantum state; and after applying a beamsplitter interaction between the first quantum state and the second quantum state and before measuring the first quantum state: first, applying a beamsplitter interaction between the second quantum state and the fourth quantum state; and second, applying a beamsplitter interaction between the first quantum state and the second quantum state.

In some embodiments, the techniques described herein relate to a system, including: a compact quantum computing system; at least one computer hardware processor; and at least one non-transitory computer readable medium storing processor-executable instructions that, when executed by the at least one computer hardware processor, cause the at least one computer hardware processor to perform a method of identifying binding configurations between a first molecular structure and a second molecular structure, the method including: generating a binding interaction graph describing possible binding interactions between portions of the first molecular structure and portions of the second molecular structure, the binding interaction graph being encoded as a graph adjacency matrix; parameterizing a multimode Gaussian state vector using the graph adjacency matrix; determining binding interactions by sampling, using holographic computing implemented on the compact quantum computing system, the multimode Gaussian state vector; and identifying, using the determined binding interactions, cliques within the binding interaction graph, the cliques corresponding to stable binding configurations between the first and second molecular structures.

In some embodiments, the techniques described herein relate to a system, wherein the method further includes identifying the first molecular structure as a candidate pharmaceutical compound.

In some embodiments, generating the binding interaction graph includes generating a binding interaction graph describing possible binding interactions between portions of the first molecular structure and portions of the second molecular structure, the second molecular structure including a structure describing a biological receptor or a portion of a biological receptor.

In some embodiments, using holographic computing implemented on the compact quantum computing system includes causing a controller to: apply a first squeezing operation to a vacuum state of a first physical qubit to generate a first quantum state in the first physical qubit; apply a second squeezing operation to a vacuum state of a second physical qubit to generate a second quantum state in the second physical qubit; cause interference between the first quantum state and the second quantum state by applying a beamsplitter interaction between the first quantum state and the second quantum state; and after applying the beamsplitter interaction: measure the first quantum state stored in the first physical qubit; and initialize a third quantum state in the first physical qubit by applying a third squeezing operation to a vacuum state of the first physical qubit.

In some embodiments, identifying the binding interactions includes identifying a first binding interaction by detecting a photon stored in the first physical qubit by measuring the first quantum state.

In some embodiments, applying the first squeezing operation includes applying a squeezing operation having squeezing parameters determined using the graph adjacency matrix.

In some embodiments, applying the first squeezing operation to the first physical qubit includes applying an electromagnetic signal to a first ancilla qubit coupled to the first physical qubit, the electromagnetic signal being configured to cause three-wave or four-wave mixing between the first quantum state and a state of the first ancilla qubit. In some embodiments, the electromagnetic signal has a frequency approximately equal to twice a resonant frequency of the first physical qubit, the first physical qubit including a first cavity resonator.

In some embodiments, applying the beamsplitter interaction further includes determining a covariance matrix using the graph adjacency matrix; and applying a beamsplitter interaction having beamsplitting parameters based on the covariance matrix.

In some embodiments, applying the beamsplitter interaction includes applying an electromagnetic signal to a second ancilla qubit coupled between the first and second physical qubits, the electromagnetic signal having a frequency approximately equal to a difference between resonant frequencies of the first and second physical qubits, the first and second physical qubits including cavity resonators.

In some embodiments, applying the beamsplitter interaction includes applying the electromagnetic signal to a transmon qubit.

In some embodiments, applying the beamsplitter interaction includes applying the electromagnetic signal to the second ancilla qubit, the electromagnetic signal configured to cause four-wave mixing between the first quantum state and the second quantum state.

In some embodiments, the method further includes causing the controller to: apply a third squeezing operation to a vacuum state of a third physical qubit to generate a fourth quantum state; and after applying the beamsplitter interaction between the first quantum state and the second quantum state and before measuring the first quantum state: first, apply a beamsplitter interaction between the second quantum state and the fourth quantum state; and second, apply a beamsplitter interaction between the first quantum state and the second quantum state.

In some embodiments, the techniques described herein relate to a method of operating a circuit quantum electrodynamics (cQED) system to execute a holographic quantum circuit, the cQED system including a first physical qubit and a second physical qubit, the method including: applying a first squeezing operation to a vacuum state of a first physical qubit to generate a first quantum state in the first physical qubit; applying a second squeezing operation to a vacuum state of a second physical qubit to generate a second quantum state in the second physical qubit; causing interference between the first quantum state and the second quantum state by applying a beamsplitter interaction between the first quantum state and the second quantum state; and after applying the beamsplitter interaction: measuring the first quantum state stored in the first physical qubit; and initializing a third quantum state in the first physical qubit by applying a third squeezing operation to a vacuum state of the first physical qubit.

In some embodiments, applying the first squeezing operation to the first physical qubit includes applying an electromagnetic signal to a first ancilla qubit coupled to the first physical qubit, the electromagnetic signal being configured to cause three-wave or four-wave mixing between the first quantum state and a state of the first ancilla qubit. In some embodiments, the electromagnetic signal has a frequency approximately equal to twice a resonant frequency of the first physical qubit, the first physical qubit including a first cavity resonator.

In some embodiments, applying the beamsplitter interaction includes applying an electromagnetic signal to a second ancilla qubit coupled between the first and second physical qubits, the electromagnetic signal having a frequency approximately equal to a difference between resonant frequencies of the first and second physical qubits, the first and second physical qubits including cavity resonators.

In some embodiments, applying the beamsplitter interaction includes applying the electromagnetic signal to a transmon qubit.

In some embodiments, applying the beamsplitter interaction includes applying the electromagnetic signal to the second ancilla qubit, the electromagnetic signal configured to cause four-wave mixing between the first quantum state and the second quantum state.

In some embodiments, the method further includes: applying a third squeezing operation to a vacuum state of a third physical qubit to generate a fourth quantum state; and after applying a beamsplitter interaction between the first quantum state and the second quantum state and before measuring the first quantum state: first, applying a beamsplitter interaction between the second quantum state and the fourth quantum state; and second, applying a beamsplitter interaction between the first quantum state and the second quantum state.

In some embodiments, the techniques described herein relate to a system, including: a compact quantum computing system including: a first physical qubit; and a second physical qubit; and at least one controller configured to execute a holographic quantum circuit using the compact quantum computing system by: applying a first squeezing operation to a vacuum state of the first physical qubit to generate a first quantum state in the first physical qubit; applying a second squeezing operation to a vacuum state of the second physical qubit to generate a second quantum state in the second physical qubit; causing interference between the first quantum state and the second quantum state by applying a beamsplitter interaction between the first quantum state and the second quantum state; and after applying the beamsplitter interaction: measuring the first quantum state stored in the first physical qubit; and initializing a third quantum state in the first physical qubit by applying a third squeezing operation to a vacuum state of the first physical qubit.

In some embodiments, applying the first squeezing operation to the first physical qubit includes applying an electromagnetic signal to a first ancilla qubit coupled to the first physical qubit, the electromagnetic signal being configured to cause three-wave or four-wave mixing between the first quantum state and a state of the first ancilla qubit. In some embodiments, the electromagnetic signal has a frequency approximately equal to twice a resonant frequency of the first physical qubit, the first physical qubit including a first cavity resonator.

In some embodiments, applying the beamsplitter interaction includes applying an electromagnetic signal to a second ancilla qubit coupled between the first and second physical qubits, the electromagnetic signal having a frequency approximately equal to a difference between resonant frequencies of the first and second physical qubits, the first and second physical qubits including cavity resonators.

In some embodiments, applying the beamsplitter interaction includes applying the electromagnetic signal to a transmon qubit.

In some embodiments, applying the beamsplitter interaction includes applying the electromagnetic signal to the second ancilla qubit, the electromagnetic signal configured to cause four-wave mixing between the first quantum state and the second quantum state.

In some embodiments, the method further includes: applying a third squeezing operation to a vacuum state of a third physical qubit to generate a fourth quantum state; and after applying a beamsplitter interaction between the first quantum state and the second quantum state and before measuring the first quantum state: first, applying a beamsplitter interaction between the second quantum state and the fourth quantum state; and second, applying a beamsplitter interaction between the first quantum state and the second quantum state.

The foregoing apparatus and method embodiments may be implemented with any suitable combination of embodiments, features, and acts described above or in further detail below. These and other aspects, embodiments, and features of the present teachings can be more fully understood from the following description in conjunction with the accompanying drawings.

### BRIEF DESCRIPTION OF DRAWINGS

Various aspects and embodiments will be described with reference to the following figures. It should be appreciated that the figures are not necessarily drawn to scale. In the drawings, each identical or nearly identical component that is illustrated in various figures is represented by a like numeral. For purposes of clarity, not every component may be labeled in every drawing.

FIG. 1A is a schematic diagram of an example of a compact quantum computing system, in accordance with some embodiments of the technology described herein.

FIG. 1B is a schematic diagram of an illustrative circuit quantum electrodynamics (cQED) system that may be used as part of the compact quantum computing system of FIG. 1A, in accordance with some embodiments of the technology described herein.

FIG. 2 is a schematic diagram of another example of a compact quantum computing system including a multi-cavity system, in accordance with some embodiments of the technology described herein.

FIG. 3A is an illustration of the crystal structure of an illustrative enzyme receptor and ligand, and the inset illustrates pharmacophores and potential binding interactions between the two molecular structures, in accordance with some embodiments of the technology described herein.

FIG. 3B is a binding interaction graph encoding the potential binding interactions between the illustrative enzyme receptor and ligand of FIG. 3A, in accordance with some embodiments of the technology described herein.

FIG. 4A is a schematic illustration of a quantum circuit for performing Gaussian boson sampling using four physical qubits.

FIG. 4B is a schematic illustration of the quantum circuit of FIG. 4A as implemented using holographic computing and a compact quantum computing system including two physical qubits, in accordance with some embodiments of the technology described herein.

FIG. 4C is a schematic diagram illustrating electromagnetic signals that may be applied to portions of a compact quantum computing system to implement the quantum circuit of FIG. 4B, in accordance with some embodiments of the technology described herein.

FIG. 5A is a schematic illustration of another quantum circuit for performing Gaussian boson sampling using four physical qubits.

FIG. 5B is a schematic illustration of the quantum circuit of FIG. 4A as implemented using holographic computing and a compact quantum computing system including three physical qubits, in accordance with some embodiments of the technology described herein.

FIG. 6A is a comparison of calculated weights of cliques identified for the illustrative enzyme receptor and ligand of FIG. 3A, the calculations performed using simulations of full Gaussian boson sampling circuits and circuits implemented using holographic computing, in accordance with some embodiments of the technology described herein.

FIG. 6B is a subgraph for the heaviest clique obtained by the calculations of FIG. 6A, in accordance with some embodiments of the technology described herein.

FIG. 6C is an illustration of binding interactions associated with the subgraph of FIG. 6B, in accordance with some embodiments of the technology described herein.

FIG. 7 is a flowchart illustrating a process of identifying binding configurations between a first molecular structure and a second molecular structure, in accordance with some embodiments of the technology described herein.

FIG. 8 is a flowchart illustrating a process of executing a holographic quantum circuit using the compact quantum computing system, in accordance with some embodiments of the technology described herein.

FIG. 9A is a schematic diagram of index combinations as perfect matchings of a graph with four nodes.

FIG. 9B is a schematic diagram illustrating construction of a 2×2 submatrix from the 8×8 symmetric matrix K=A^{⊕2} for two photons measured in the last two output modes of a Gaussian boson sampling circuit, in accordance with some embodiments of the technology described herein.

FIG. 10 is a schematic diagram of an illustrative implementation of a computer system that may be used in connection with some embodiments of the technology described herein.

### DETAILED DESCRIPTION

In the field of molecular modeling, molecular docking techniques include methods to predict the preferred orientations of one molecular structure relative to another molecular structure when bound to form a stable complex. Molecular docking techniques may be used, for example, to predict stable binding configurations between molecular structures being considered for pharmaceutical applications (e.g., small molecule ligands) and respective biological structures (e.g., biological receptors, proteins, peptides, nucleic acids, lipids, etc.). While molecular docking techniques are particularly useful for structure-based pharmaceutical and material discovery, the techniques are not limited to predicting only biological or pharmaceutical molecular interaction problems, as aspects of the technology described herein are not limited in this respect.

However, due to the complex nature of large molecular structures, including proteins and other biological targets, molecular docking simulations are challenging if not impossible to perform using classical computing systems and algorithms. For example, several classes of molecular docking problems require the calculation of permanents, which is a problem known to be #P-hard, escalating to #P-complete problems for binary matrices. Such complexity therefore places these classes of problems beyond the reach of conventional, classical computing techniques for sufficiently large matrices and correspondingly more complex simulations.

Quantum computers that implement boson sampling offer a promising solution for such complex problems, however. Boson sampling techniques involve sampling photons scattered by an N-mode linear interferometer. The distribution of measured outcomes is defined in terms of the permanent of the N×N matrix of transition probability amplitudes that define the linear interferometer. As a result, boson sampling techniques enable the sampling of bosons from a distribution that would be challenging or impossible to simulate classically for large values of N.

The inventors have recognized and appreciated that circuit quantum electrodynamic (cQED) quantum computing architectures provide an improved platform for the implementation of boson sampling. While optical photons are conventionally used to implement boson sampling, the measurement of optical photons is difficult to achieve with near-unit efficiency. In contrast, cQED architectures utilize microwave photons stored and retrieved from, for example, microwave cavity resonators (e.g., a simple harmonic oscillator with equidistant level spacing). The use of microwave photons and microwave cavity resonators enables accurate photon number measurements that are not possible using optical photons such that boson sampling techniques may be more accurately implemented using cQED architectures. Boson sampling, implemented using cQED techniques, may therefore be used to accelerate molecular docking simulations by sampling from the space of possible molecular configurations.

The inventors have further recognized and appreciated that boson sampling computations, when parameterized for implementation using conventional quantum circuit techniques, may be too lengthy to perform on current quantum computing platforms. For example, the example molecular docking problem described herein is estimated, for IBM's Guadalupe v2 system, to require a quantum circuit depth (e.g., a length of the quantum algorithm) of 14573 circuit layers after transpiling. However, it is estimated that IBM's Guadalupe v2 system can only practically perform quantum circuits with a circuit depth less than 100.

Accordingly, the inventors have developed techniques for performing boson sampling using holographic quantum computing techniques as implemented on compact quantum computing systems. In holographic quantum computing, a complex quantum circuit using multiple physical qubits may be rewritten (e.g., “linearized”) so that it may be implemented using fewer physical qubits. The rewritten quantum circuit may be implemented using simplified hardware by repurposing physical qubits to store or manipulate different quantum information throughout the course of the quantum circuit. For example, a physical qubit may be used to store and/or operate on a first quantum state, the first quantum state may be measured, and then the physical qubit may be reinitialized with a second quantum state for use in the next portion of the quantum circuit. In this manner, smaller quantum computing devices (e.g., having two or three physical qubits) may be used to implement complex quantum circuits, including boson sampling circuits.

By measuring and reinstantiating quantum states within quantum modes throughout the quantum circuit, rather than at the end of the quantum circuit, longer quantum circuits may be implemented. In particular, because the individual quantum states are sampled more frequently and are therefore less likely to decohere prior to measurement. Within the context of molecular docking techniques, the use of holographic quantum computing enables the sampling of larger state vectors—and therefore the solving of larger molecular docking problems—than conventional quantum circuit design.

Additionally, holographic quantum computing techniques can be implemented using compact quantum computing systems (e.g., systems having a fewer number of qubits than quantum modes to be instantiated during the length of the quantum circuit). Compact quantum computing systems require less hardware complexity (e.g., requiring fewer total qubits, ancilla qubits, and coupling devices) to implement but, when used with holographic quantum computing techniques, do not sacrifice computing complexity. The techniques described herein therefore represent an improvement to quantum computing techniques, allowing for longer quantum circuits to be implemented without loss of coherence and using fewer hardware components to implement said quantum circuits.

In some embodiments, the techniques described herein include systems and methods for identifying, using a compact quantum computing system, binding configurations between a first molecular structure and a second molecular structure. As one example, the first molecular structure may be a potential pharmaceutical compound (e.g., a small molecule ligand), and the second molecular structure may be a biological target (e.g., a receptor). The techniques include generating a binding interaction graph describing possible and/or compatible binding interactions between portions (e.g., pharmacophores) of the first molecular structure and the second molecular structure. The binding interaction graph may be encoded mathematically as a graph adjacency matrix, with entries of the graph adjacency matrix indicating whether pairs of interactions are compatible (e.g., can be established simultaneously). The graph adjacency matrix may further be weighted (e.g., using a weighting vector) based on the intermolecular interactions involved in each pair of interactions (e.g., based on binding potentials or other chemical parameters).

In some embodiments, the graph adjacency matrix may be used to parameterize a multimode Gaussian state vector. The Gaussian state vector may then be used to prepare a holographic matrix product state (MPS) circuit for execution by a compact quantum computing system. Thereafter, binding interactions between portions of the first and second molecular structure may be identified by sampling the multimode Gaussian state vector (e.g., by executing the holographic MPS circuit using the compact quantum computing system).

In some embodiments, after identifying the binding interactions, one or more cliques within the binding interaction graph may be identified, where “cliques” describe stable binding configurations between the first and second molecular structure. In particular, the identified cliques may be used to solve the maximum clique problem, in which the largest fully connected subgraph within the binding interaction graph, representing the largest set of compatible binding interactions, is identified.

In some embodiments, techniques for executing a holographic quantum circuit using a compact quantum computing system are provided herein. In some embodiments, the compact quantum computing system may be a cQED system having a number of physical qubits that is smaller than a number of boson modes to be sampled by execution of the holographic quantum circuit. The techniques include applying a first squeezing operation to a vacuum state of a first physical qubit to generate a first quantum state in the first physical qubit. Additionally, a second squeezing operation is applied to a vacuum state of a second physical qubit to generate a second quantum state in the second physical qubit. The first and/or second squeezing operations may be applied by, for example, applying an electromagnetic signal (e.g., a microwave signal) to an ancilla qubit coupled to the first or second physical qubit, respectively.

In some embodiments, after applying the first and/or second squeezing operations, a beamsplitter interaction may be applied to the compact quantum computing system in order to cause interference between the first quantum state and the second quantum state. For example, the beamsplitter interaction may be applied by applying an electromagnetic signal (e.g., a microwave signal) to an ancilla qubit or a coupling device coupled between the first and second physical qubits. After applying the beamsplitter interaction, the first quantum state stored in the physical qubit is measured to sample a first portion of the Gaussian state vector. Thereafter, a third quantum state is initialized in the first physical qubit by applying a third squeezing operation to a vacuum state of the first physical qubit.

Following below are more detailed descriptions of various concepts related to, and embodiments of, techniques for coupling quantum modes using a linear inductive coupler. It should be appreciated that various aspects described herein may be implemented in any of numerous ways. Examples of specific implementations are provided herein for illustrative purposes only. In addition, the various aspects described in the embodiments below may be used alone or in any combinations and are not limited to the combinations explicitly described herein.

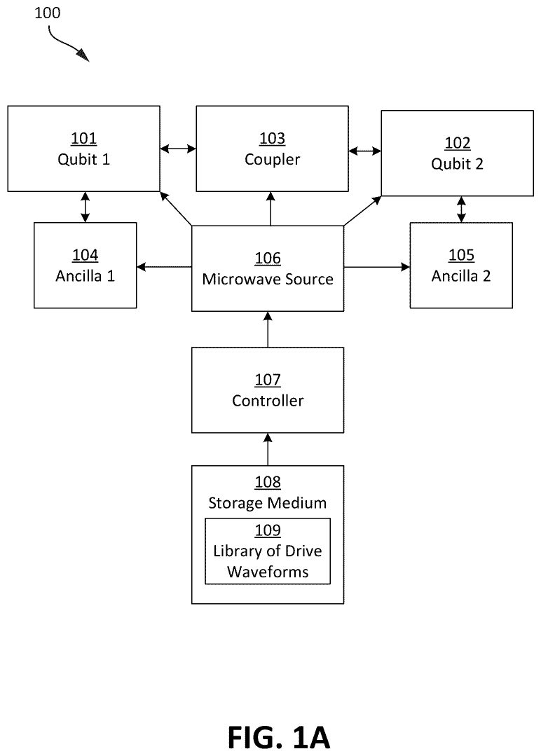

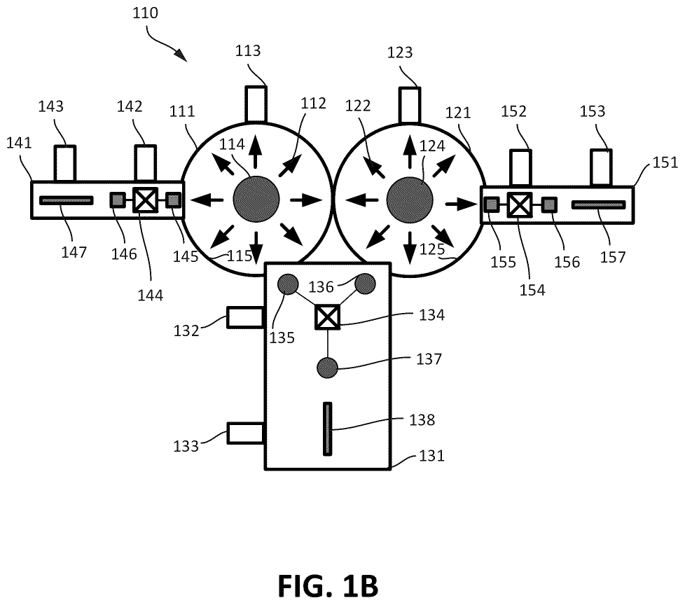

FIG. 1A is a schematic diagram of a quantum system 100, according to some embodiments. The quantum system 100 includes a first physical qubit 101, a second physical qubit 102, a coupler 103, a first ancilla qubit 104, a second ancilla qubit 105, and a microwave source 106. The microwave source 106 may be communicatively coupled to a controller 107 (e.g., comprising at least one computer hardware processor) arranged to access one or more drive waveforms from library 109 stored on a computer-readable storage medium 108. While only two physical qubits, one coupler, and two ancilla qubits are shown in the example quantum system 100 of FIG. 1A, other embodiments may include additional physical qubits, couplers, and/or ancilla qubits.

In some embodiments, the first physical qubit 101 and the second physical qubit 102 may be any type of cavity resonator configured to support a quantum states (e.g., to support photons). For example, in some embodiments, the first physical qubit 101 and the second physical qubit 102 may be a transmission line resonator or a three-dimensional microwave resonator cavity. In some embodiments, the three-dimensional microwave resonator cavity may be formed from a superconducting material, such as aluminum.

The coupler 103 may be configured to provide switchable beamsplitter interactions between quantum states stored in the first physical qubit 101 and the second physical qubit 102. For example, the coupler 103 may actuate Hamiltonians of the form H=g(a₁^{†}a₂+a₁a₂^{†}) between quantum states stored in the first physical qubit 101 and the second physical qubit 102. The coupler 103 may be implemented using, for example, a superconducting circuit including but not limited to a transmon qubit, a superconducting nonlinear asymmetric inductive element (SNAIL), a flux-pumped DC superconducting quantum interference device (SQUID), and/or any suitable parametric coupler. The coupler 103 may be arranged to implement a beamsplitter interaction between quantum states stored in the first physical qubit 101 and the second physical qubit 102 using, for example, four-wave mixing and/or three-wave mixing processes.

In some embodiments, the first ancilla qubit 104 and the second ancilla qubit 105 may be dispersively coupled to the first physical qubit 101 and the second physical qubit 102, respectively. Each ancilla qubit 104, 105 may be coupled to a single physical qubit of the quantum system 100. The ancilla qubits 104, 105 may be any suitable quantum system having three distinct states (e.g., a multi-state quantum system), such as but not limited to, those based on a superconducting Josephson junction such as a charge qubit (Cooper-pair box), a flux qubit or a phase qubit, a transmon qubit, or combinations thereof. The ancilla qubits 104, 105 may be controlled (e.g., by microwave signals generated by microwave source 106) to implement rotations of quantum states of the ancilla qubits 104, 105 themselves. Additionally, the ancilla qubits 104, 105 may be controlled to interact with the associated physical qubits 101 and 102, respectively, to implement controlled interactions between the quantum states stored in the physical qubits 101 and 102 and the respective states of the ancilla qubits 104, 105.

In some embodiments, the microwave source 106 may be coupled to the first physical qubit 101, the second physical qubit 102, the first ancilla qubit 104, the second ancilla qubit 105, and/or the coupler 103. The coupling between the microwave source 106 and the other components of the quantum system 100 provides a way for the microwave source to apply microwave radiation to each of the components of the quantum system 100. In some embodiments, the microwave source 106 may be capacitively coupled to each of the components and may be configured to apply one or more electromagnetic signals (e.g., microwave drive waveforms) to the coupled components. In some embodiments, the microwave source 106 may be multiple microwave sources.

In some embodiments, a library of precomputed drive waveforms may be stored on a computer readable storage medium and accessed in order to apply said waveforms to one or more components of the quantum system 100. For example, controller 107 may access the library of drive waveforms 109 stored on storage medium 108 (e.g., in response to user input provided to the controller) and control the microwave source 106 to apply drive waveforms to one or more components of the quantum system 100.

As used herein, application of such an electromagnetic signal or pulse may also be referred to as “driving” of the ancilla qubit and/or the physical qubit. Coupling may utilize any technique(s) to couple the ancilla qubit and the physical qubit, such as by coupling electric and/or magnetic fields generated by the ancilla qubit and the physical qubit. According to some embodiments, the ancilla qubit (e.g., a transmon) may be coupled to the physical qubit, being a mechanical resonator, via a piezoelectric coupling. According to some embodiments, the ancilla qubits 104, 105 may be coupled to the physical qubits 101 and 102, being a magnetic resonator, by coupling the ancilla qubit (e.g., a transmon) to phonons, which in turn couple to magnons via magnetostrictive coupling.

FIG. 1B is a schematic diagram of hardware components of an illustrative quantum system 110 that may be used as part of the quantum system 100 of FIG. 1A (for simplicity the microwave source 106, controller 107, and storage medium 108 are not shown in the example of FIG. 1B), according to some embodiments. The quantum system 110 includes a first three-dimensional (3D) cavity 111, a second 3D cavity 121, a coupling device 131, a first ancilla device 141, and a second ancilla device 151.

In some embodiments, the first and second 3D cavities 111 and 121 each act as a 3D version of a λ/4 transmission line resonator between central stubs 114 and 124, respectively, and outer walls 115 and 125, respectively. For example, the diameter of central stubs 114 and 124 may be 3.2 mm and the diameter of the outer walls 115 and 125 may be 9.5 mm. It is noted, however, that embodiments are not limited to any particular dimensions. The resonant frequency of each of the physical qubits 101 and 102 may be determined by the height of the central stub 114 and 124 within their respective cavity. For example the central stub 114 may have a height of 4.8 mm and the second central stub 124 may have a height of 5.6 mm. The first 3D cavity 111 supports microwave radiation 112 of a first frequency and the second 3D cavity 121 supports microwave radiation 122 of a second frequency that is different from the first frequency. In some embodiments, the first cavity 111 and the second cavity 121 include ports 113 and 123, respectively, through which microwave radiation from the microwave source 106 may be applied. Applying microwave radiation to a cavity may, for example, implement a displacement operation on the quantum state of the cavity.

In some embodiments, the coupling device 131 includes a coupling transmon 134 that provides a nonlinear interaction between the first cavity 111 and the second cavity 121. The transmon 134 is coupled to a first antenna 135 that is inserted at least partially into the first cavity 111 and a second antenna 136 that is inserted at least partially into the second cavity 121 such that at least a portion of each antenna protrudes into its respective cavity. The first and second antennas 135/136 may be, for example, circular pads that provide capacitive coupling to the first and second cavities 111/121, respectively.

In some embodiments, the coupling device 131 also includes a resonator 138 that provides the ability to readout the state of the transmon 134. A third antenna 137 couples the resonator 138 to the resonator 138. In some embodiments, the resonator 138 is a quasi-planar resonator (e.g., a stripline resonator) with a lower Q value than either the first cavity 111 or the second cavity 121. In some embodiments, the transmon 134 and the resonator 138 are fabricated on a single sapphire substrate. A readout pulse of microwave radiation may be received by a pump port 132 and a resulting microwave signal may be received from readout port 133.

In some embodiments, the nonlinearity of the transmon 134 of the coupling device 131 enables four-wave mixing, which is used to perform a frequency-converting bilinear coupling between the first cavity 111 and the second cavity 121. The four-wave mixing is controlled by pumping the transmon 134 via a pump port 132 with microwave radiation that satisfies the frequency matching condition ω₁−ω₂=ωₚ₂−ωₚ₁, where ω₁ is the resonant frequency of the first cavity 111, ω₂ is the resonant frequency of the second cavity 121, ωₚ₁ is the frequency of the first pump associated with a mode c, and ωₚ₂ is the frequency of the second pump associated with a mode d. This coupling implements an effective time-dependent beamsplitter interaction between the cavity modes. As is known from conventional optics, the unitary evolution of the beam splitter is described by the unitary operator:

$${U}_{BS} ( θ ) = {e}^{- i {\int }_{0}^{t} {H}_{BS} ( τ ) d τ} = {e}^{i θ ( {a}^{†} b + {ab}^{†} )} ,$$

$$where$$

$${H}_{BS} ( τ ) = gt ( τ ) ( {a}^{†} b + {ab}^{†} ) ,$$

$$and$$

$$θ = {\int }_{0}^{t} g ( τ ) d τ .$$

For θ=π/2, the beam splitter unitary operator implements the SWAP operation that exchanges the states between the two cavity modes associated with the annihilation operators a and b, respectively. For θ=π/4 and θ=−π/4 the unitary operator corresponds to a 50/50 beamsplitter. The beamsplitter interaction may accordingly be tuned using the parameter θ.

In some embodiments, first ancilla device 141 is similar to the coupling device 131, but only couples to the first cavity 111, not both cavities. The first ancilla device includes a pump port 142 for driving a transmon 144 with pump and readout pulses of microwave radiation and a readout port 143 for receiving readout microwave signals from the transmon 144. The transmon 144 is coupled to the first cavity 111 via a first antenna pad 145 that at least partially protrudes into the first cavity 111. A second antenna pad 146 couples the transmon 144 to a quasi-planar resonator 147.

In some embodiments, the second ancilla device 151 is similar to the first ancilla device 141, but is coupled to only the second cavity 121, not the first cavity 111. The second ancilla device includes a pump port 152 for driving a transmon 154 with pump and readout pulses of microwave radiation and a readout port 153 for receiving readout microwave signals from the transmon 154. The transmon 154 is coupled to the first cavity 111 via a first antenna pad 155 that at least partially protrudes into the first cavity 111. A second antenna pad 156 couples the transmon 144 to a quasi-planar resonator 147.

In some embodiments, the first ancilla device 141 and the second ancilla device 151 may be used to control and/or measure quantum states stored in the cavities 111 and 121, respectively. For example, quantum states may be instantiated in either of the cavities 111 or 121 by applying a squeezing operation to the cavities 111 or 121 via the respective ancilla devices 141 or 151. The squeezing operations may be implemented by driving an ancilla device 141 or 151 with an electromagnetic signal having a frequency approximately equal to twice a resonant frequency of the respective cavity 111, 121.

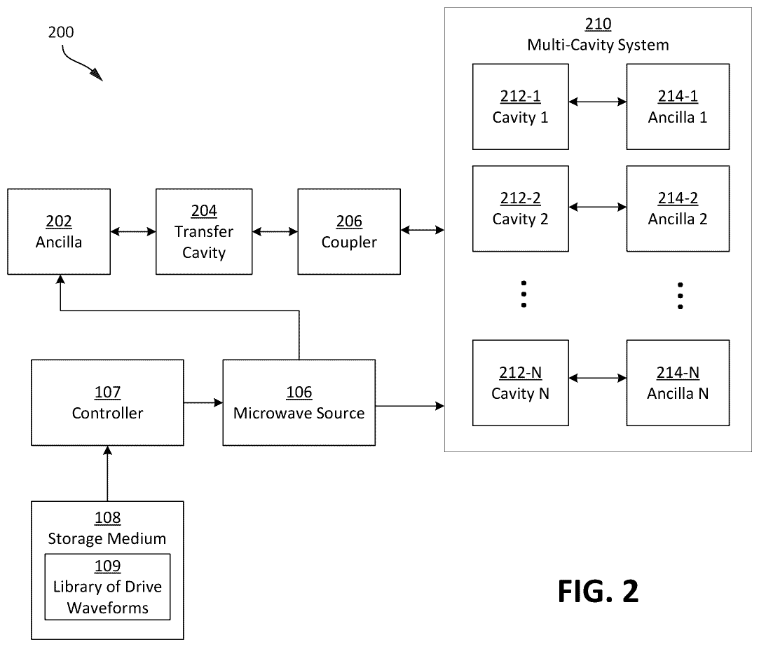

FIG. 2 is a schematic diagram of another example of a compact quantum computing system 200 including a multi-cavity system 210, in accordance with some embodiments of the technology described herein. The multi-cavity system 210 includes multiple cavities 212-1 through 212-N (e.g., microwave cavity resonators) and respective ancilla qubits 214-1 through 214-N each coupled to one of the cavities 212-1 through 212-N. The ancilla qubits 214-1 through 214-N may be, for example, transmon qubits The multi-cavity system 210 may be, for example, a single piece of superconducting material (e.g., aluminum) with each of the cavities 212-1 through 212-N formed integrally (e.g., by machining or other techniques). The ancilla qubits 214-1 through 214-N may be integrated into the multi-cavity system 210 or may be coupled from an external position. The ancilla qubits 214-1 through 214-N may be configured to, for example, manipulate quantum states stored in the cavities 212-1 through 212-N (e.g., by applying single-qubit quantum gates, by measuring the quantum states, and/or by instantiating the quantum states).

In some embodiments, the compact quantum computing system 200 also includes an external ancilla qubit 202 coupled to the multi-cavity system 210 by a transfer cavity 204 and a coupler 206. The ancilla qubit 202 may be, for example, a charge qubit (Cooper-pair box), a flux qubit or a phase qubit, a transmon qubit, or combinations thereof. The transfer cavity 204 may be, for example, a microwave cavity resonator configured to store a quantum state instantiated by the ancilla qubit 202.

In some embodiments, the coupler 206 may be a parametric coupler configured to couple pairs of quantum states stored in cavities 212-1 through 212-N or to couple a quantum state stored in one of the cavities 212-1 through 212-N to a quantum state stored in the transfer cavity 204. The coupler 206 may be, for example, any suitable parametric coupler including but not limited to a SNAIL or SQUID.

In some embodiments, the compact quantum computing system 200 may include a microwave source 106 coupled to the external ancilla qubit 202 and the multi-cavity system 210. The microwave source 106 may be configured to apply microwave signals to one or more of the external ancilla qubit 202, the cavities 212-1 through 212-N, and/or the ancilla qubits 214-1 through 214-N to cause the application of quantum operations to respective components of the compact quantum computing system 200.

In some embodiments, a library of precomputed drive waveforms may be stored on a computer readable storage medium and accessed in order to apply said waveforms to one or more components of the quantum system 100. For example, controller 107 may access the library of drive waveforms 109 stored on storage medium 108 (e.g., in response to user input provided to the controller) and control the microwave source 106 to apply drive waveforms to one or more components of the quantum system 100.

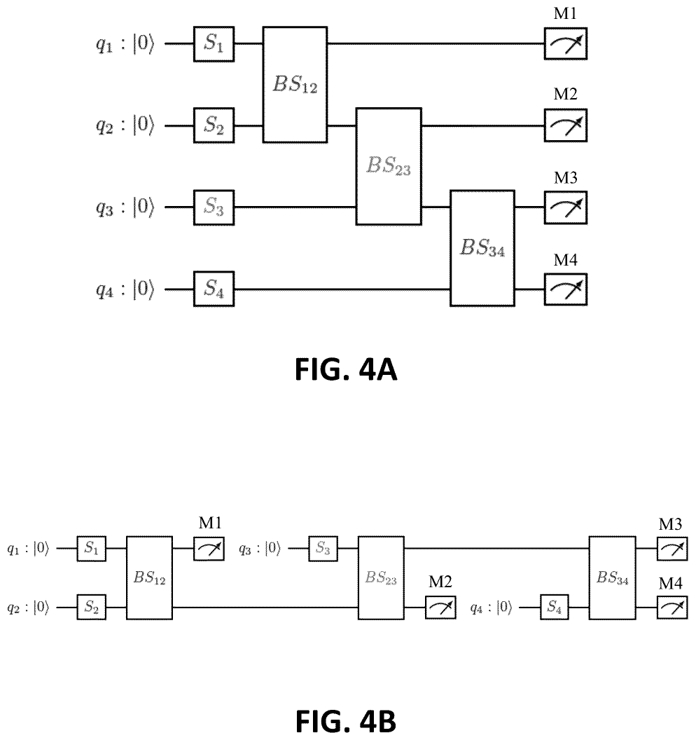

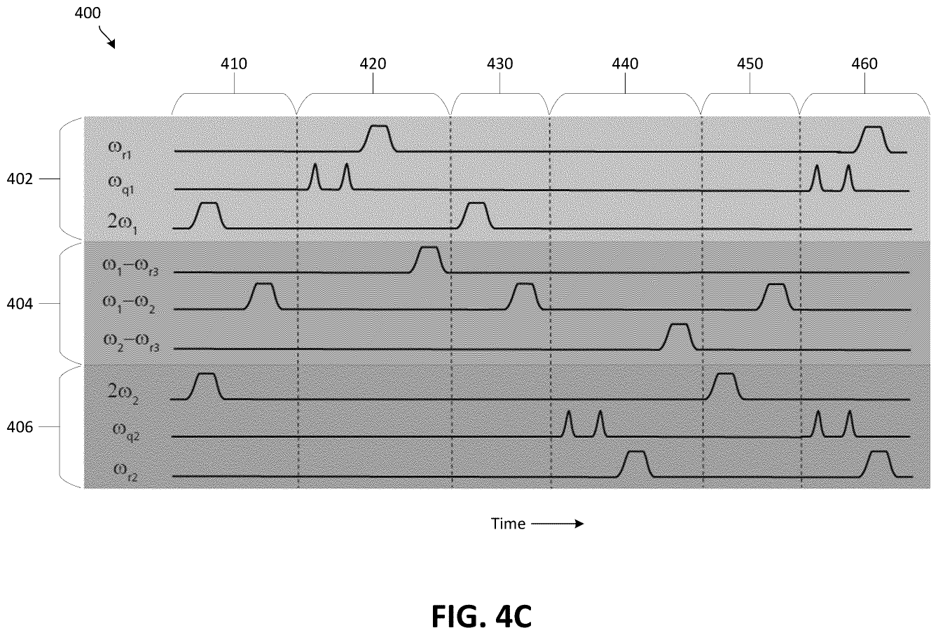

FIG. 4A illustrates a quantum circuit diagram for performing Gaussian boson sampling and/or molecular docking simulations. The four lines represent quantum states stored in four physical qubits (e.g., four cavity resonators) and the diagram, from left to right, indicates operations performed on each of the quantum states as time progresses. To perform Gaussian boson sampling using a four qubit system, each qubit starts in a vacuum state, |0 [symbol] and is initialized by a squeezing operation, S. Thereafter, a sequence of beamsplitter interactions, BS, are applied between pairs of the four qubits. At the end, each quantum state is measured (M1-M4) to determine a number of photons stored in each qubit.

The quantum circuit of FIG. 4A requires the use of four physical qubits and associated hardware including ancillae, coupling devices, microwave inputs and readouts, etc. However, linearizing the quantum circuit using holographic quantum computing substantially reduces the required hardware complexity. FIG. 4B illustrates such a quantum circuit diagram for performing Gaussian boson sampling and/or molecular docking simulations, in accordance with some embodiments described herein.

The quantum circuit of FIG. 4B is configured to be implemented using a two-mode quantum system (e.g., as described in connection with FIGS. 1A and 1B). Two quantum states, q₁ and q₂, are initialized in two physical qubits (e.g., cavity resonators) using associated squeezing operations, S₁ and S₂. A first beamsplitter interaction, BS₁₂, is then implemented between the two quantum states q₁ and q₂ stored in the two physical qubits. The first quantum state q₁ may then be measured to obtain measurement M1. Thereafter, a third quantum state q₃ may be initialized in the original first physical qubit. This process may be iterated until the entire computation is completed.

FIG. 4C is a schematic pulse diagram 400 illustrating electromagnetic signals that may be applied to portions of a compact quantum computing system (e.g., the systems of FIGS. 1A-1B) to implement the quantum circuit of FIG. 4B, in accordance with some embodiments of the technology described herein. The pulse diagram 400 illustrates the application of the electromagnetic signals with time increasing from left to right. The top three signals in section 402 may be applied to an ancilla qubit (e.g., devices 104 or 141 of FIG. 1A or 1B, respectively) coupled to a first physical qubit (e.g., physical qubits 101 or 111 of FIG. 1A or 1B, respectively). The middle three signals in section 404 may be applied to a coupling device (e.g., devices 103 or 131 of FIG. 1A or 1B, respectively) coupled between two physical qubits. The lower three signals in section 406 may be applied to an ancilla qubit (e.g., devices 105 or 151 of FIG. 1A or 1B, respectively) coupled to a second physical qubit (e.g., physical qubits 102 or 121 of FIG. 1A or 1B, respectively).

The pulse diagram 400 includes, for each signal line, a notation at left indicating the frequency of each electromagnetic signal applied to the compact quantum computing system. In section 402, the signal lines correspond, from top to bottom, to electromagnetic signals applied to the first ancilla qubit and having frequencies approximately equal to the resonant frequency of a first readout resonator coupled to the first ancilla qubit, ωᵣ₁, approximately equal to the resonant frequency of the first ancilla qubit, ω_{q1}, and approximately equal to twice the resonant frequency of the first physical qubit, 2ω₁. In section 404, the signal lines correspond, from top to bottom, to electromagnetic signals applied to the coupling device and having frequencies approximately equal to a difference between the resonant frequency of the first physical qubit and the resonant frequency of the readout resonator coupled to the coupling device, ω₁−ω₁₃, approximately equal to a difference between the resonant frequencies of the first and second physical qubits, ω₁−ω₂, and approximately equal to a difference between the resonant frequency of the second physical qubit and the resonant frequency of the readout resonator coupled to the coupling device, ω₂−ωᵣ₃. In section 406, the signal lines correspond, from top to bottom, to electromagnetic signals applied to the second ancilla qubit and having frequencies approximately equal to twice the resonant frequency of the second physical qubit, 2ω₂, approximately equal to the resonant frequency of the second ancilla qubit, ωq₂, and approximately equal to the resonant frequency of a second readout resonator coupled to the first ancilla qubit, ωᵣ₂.

In some embodiments, the pulse diagram 400 begins with the application of squeezing and beamsplitting operations in portion 410. The electromagnetic signals of sections 402 and 406 in portion 410 correspond to the application of squeezing operations to each of the physical qubits, the squeezing operations configured to initialize quantum states in each of the physical qubits. The squeezing operation may be implemented by the application of single electromagnetic pulses (e.g., microwave pulses) to each of the ancilla qubits. The beamsplitting operation may be implemented by the application of a single electromagnetic pulse (e.g., a microwave pulse) to the coupling device after the application of the squeezing operations. The electromagnetic signal implementing the beamsplitting operation may have a frequency configured to cause three-wave or four-wave mixing between the two physical qubits.

In some embodiments, the pulse diagram 400 may then proceed to measuring and resetting the first physical qubit in portion 420. The first physical qubit may be measured by applying, to the first ancilla qubit, two electromagnetic pulses having a frequency approximately equal to ω_{q1} followed by the application of an electromagnetic pulse having a frequency approximately equal to ωᵣ₁, causing readout from the first physical qubit and measurement of mode M1 as depicted in FIG. 4B. The state of the first physical qubit may then be reset by applying an electromagnetic pulse to the coupling device.

In some embodiments, the pulse diagram 400 may then proceed to a second set of squeezing and beamsplitting operations in portion 430. The squeezing operation may be implemented by applying an electromagnetic signal to the first ancilla qubit and may be configured to initialize a new quantum state in the first physical qubit. As in portion 410, the beamsplitting operation may be implemented by applying an electromagnetic signal to the coupling device and may be configured to cause interference between the quantum states stored in the first and second physical qubits.

In some embodiments, the pulse diagram 400 may then proceed to measuring and resetting the second physical qubit in portion 440. The second physical qubit may be measured by applying, to the second ancilla qubit, two electromagnetic pulses having a frequency approximately equal to ω_{q2} followed by the application of an electromagnetic pulse having a frequency approximately equal to ωᵣ₂, causing readout from the second physical qubit and measurement of mode M2 as depicted in FIG. 4B. The state of the second physical qubit may then be reset by applying an electromagnetic pulse to the coupling device.

In some embodiments, the pulse diagram 400 may then proceed to a third set of squeezing and beamsplitting operations in portion 450. The squeezing operation may be implemented by applying an electromagnetic signal to the second ancilla qubit and may be configured to initialize a new quantum state in the second physical qubit. As in portions 410 and 430, the beamsplitting operation may be implemented by applying an electromagnetic signal to the coupling device and may be configured to cause interference between the quantum states stored in the first and second physical qubits.

In some embodiments, the pulse diagram 400 may then proceed to measurements of both physical qubits in portion 460. The measurements of each quantum state may proceed as described in connection with portions 420 and 440 but may be performed concurrently. The measurements of portion 460 correspond to the measurements of modes M3 and M4 as depicted in FIG. 4B.

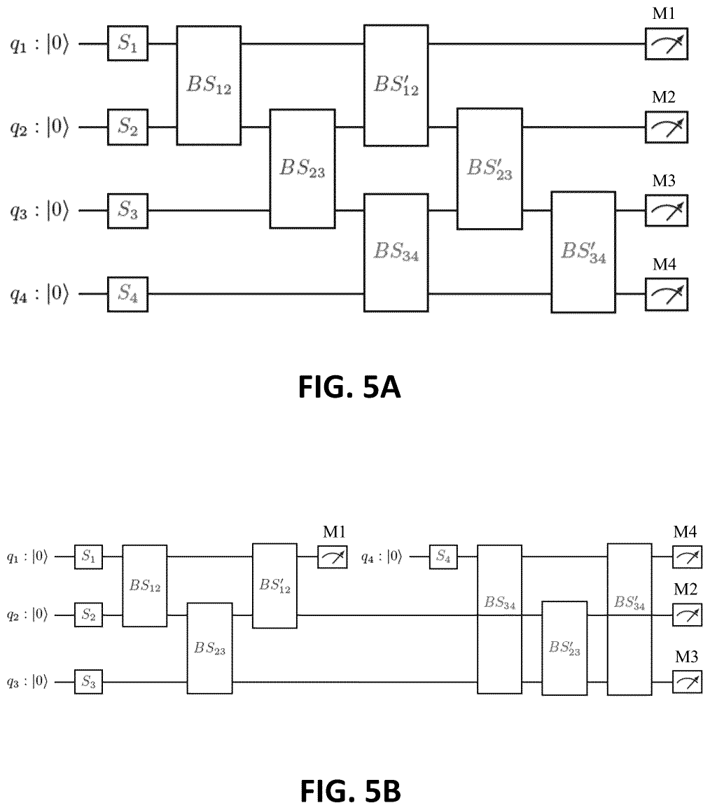

In some embodiments, the quantum circuits of FIGS. 4A and 4B may be implemented using a compact quantum system having three physical qubits (e.g., a system like that of FIG. 1B but including an additional cavity resonator, coupling ancilla, and/or an additional a readout cavity, or a multi-cavity system like that of FIG. 2). FIG. 5A illustrates the same quantum circuit diagram of FIG. 4A with two layers of beamsplitters. The circuit of FIG. 5A may be linearized using holographic quantum computing techniques, as depicted in the example of FIG. 5B, where three physical qubits are used to implement the circuit.

In some embodiments, the quantum circuits of FIGS. 4B and/or 5B may be executed to perform simulations of molecular docking, which predict noncovalent interactions between two molecules (e.g., a pharmaceutical molecule and a biological target such as a receptor). Orientations of the two molecules of interest may be represented by a graph, where vertices correspond to pairs of interactions between pharmacophores, and the edges connect pairs of interaction with compatible Euclidean distances between atomic interactions. This graph may be represented by a weighted adjacency matrix, with elements indicating whether a particular interaction is compatible and values corresponding to a strength of that interaction.

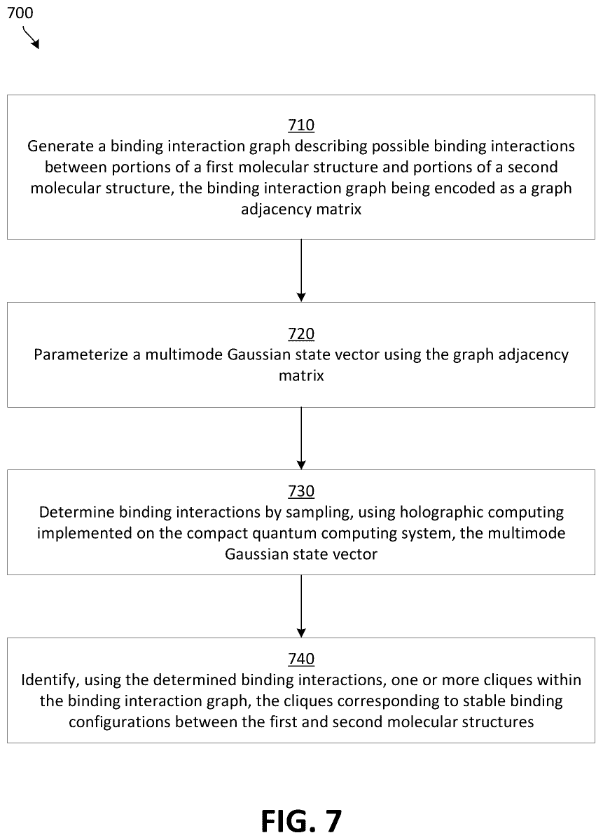

FIG. 7 is a flowchart illustrating a process 700 of identifying binding configurations between a first molecular structure and a second molecular structure, in accordance with some embodiments of the technology described herein. Portions of process 700 may be performed by any suitable classical computing device or devices, as aspects of the technology described herein are not limited in this respect. For example, portions of process 700 may be performed by a classical computing device that is part of a compact quantum computing system. In other embodiments, portions of process 700 may be performed by one or more classical computing devices external to the compact quantum computing system.

In some embodiments, process 700 begins at act 710, where a binding interaction graph may be generated. The binding interaction graph may describe possible binding interactions between portions of the first molecular structure and portions of the second molecular structure. In some embodiments, the first molecular structure may be a target pharmaceutical compound (e.g., a ligand or other small molecule) and the second molecular structure may be a biological target (e.g., a receptor, protein, or other biological molecular structure).

In some embodiments, the binding interaction graph may be encoded as a graph adjacency matrix. The graph adjacency matrix, A, may be an M×M matrix, where M is the total number of possible interactions between the first and second molecular structures. Entries, Aᵢⱼ, of the graph adjacency matrix may be equal to one where the interactions i and j are compatible and may otherwise be equal to zero.

In some embodiments, the graph adjacency matrix may further be modified by a weighting vector. The weighting vector may be arranged to weight the compatible interactions between portions of the first and second molecular structures based on interaction strengths between the relevant portions of the first and second molecular structures (e.g., based on binding affinity or other aspects of intermolecular interactions), as described in the section titled “Interaction Graph” herein.

In some embodiments, after act 710, the process 700 may proceed to act 720, in which a multimode Gaussian state vector may be parameterized using the graph adjacency matrix. For example, the graph adjacency matrix, A, may be used to encode the graph problem (e.g., of molecular docking) into Gaussian quantum states, as described in the section titled “Multimode Gaussian of Bosonic Modes” herein. In particular, the graph adjacency matrix, A, may be encoded into a covariance matrix used to define parameters of the operations to be implemented on the compact quantum computing system.

In some embodiments, after act 720, the process 700 may proceed to act 730, in which binding interactions may be determined by sampling, using holographic computing techniques implemented on a compact quantum computing system (e.g., a quantum computing system having fewer physical qubits than Gaussian modes to be sampled), the multimode Gaussian state vector. To sample the multimode Gaussian state vector using holographic computing techniques, a series of quantum operations may be performed using the compact quantum computing system as described in connection with FIGS. 4A-5B and FIG. 8 herein. In some embodiments, identifying the binding interactions may include, when measuring a quantum state, detecting a photon stored in a physical qubit.

In some embodiments, after act 730, the process 700 may proceed to act 740, in which one or more cliques within the binding interaction graph may be identified using the determined binding interactions. The cliques may correspond to stable binding configurations between two or more compatible binding pairs of the first and second molecular structures, as described in the section titled “Molecular Docking Mapped as a Graph Search Problem” herein. In some embodiments, a clique or cliques with larger sampling weights may be identified as being the most stable molecular configuration(s) for the first and second molecular structures.

In some embodiments, the method may further comprise identifying the first molecular structure as a candidate pharmaceutical compound (e.g., based on the identified cliques and/or binding interactions). The method may include manufacturing the first molecular structure and may further include determining safety and/or efficacy of the first molecular structure by performing a clinical and/or nonclinical study using the first molecular structure.

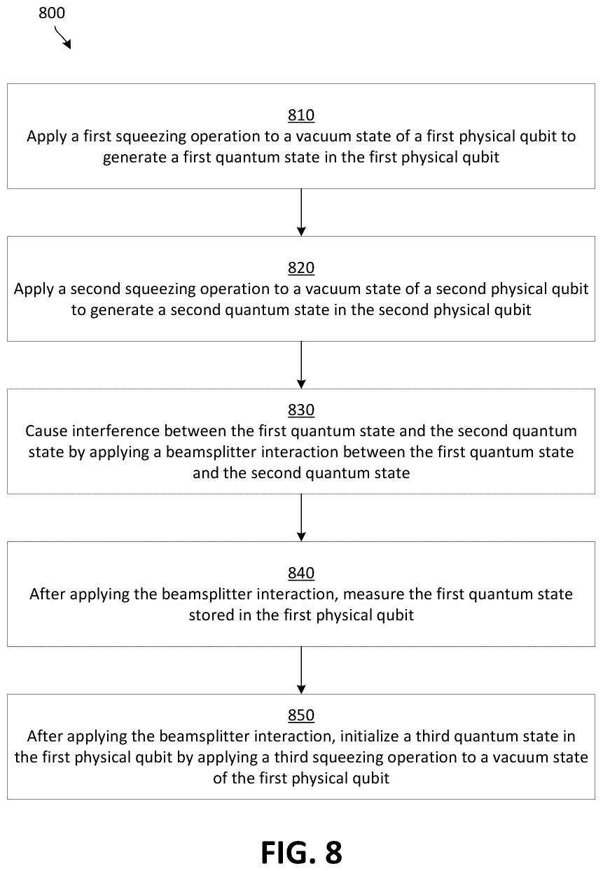

FIG. 8 is a flowchart illustrating a process 800 of executing a holographic quantum circuit using a compact quantum computing system, in accordance with some embodiments of the technology described herein. Process 800 may be performed using any suitable compact quantum computing system. For example, process 800 may be performed using one or more of the compact cQED quantum computing systems as described in connection with FIGS. 1A-1B and/or 2 herein.

In some embodiments, the process 800 may begin at act 810, in which a first squeezing operation may be applied to a vacuum state of a first physical qubit to generate a first quantum state in a first physical qubit. The squeezing operation may be implemented, for example, by applying an electromagnetic signal to a first ancilla qubit coupled to the first physical qubit (e.g., to cause three- or four-wave mixing between the first ancilla qubit and the first physical qubit). The electromagnetic signal may be, for example, a microwave signal having a frequency approximately equal to twice the resonant frequency of the first physical qubit. In some embodiments, parameters of the first squeezing operation may be determined based on a graph adjacency matrix (e.g., in the context of a graph-based simulation problem).

In some embodiments, after act 810, the process 800 may proceed to act 820 in which a second squeezing operation may be applied to a vacuum state of a second physical qubit to generate a second quantum state in the second physical qubit. The squeezing operation may be implemented, for example, by applying an electromagnetic signal to a second ancilla qubit coupled to the second physical qubit (e.g., to cause three- or four-wave mixing between the second ancilla qubit and the second physical qubit). The electromagnetic signal may be, for example, a microwave signal having a frequency approximately equal to twice the resonant frequency of the second physical qubit. In some embodiments, parameters of the second squeezing operation may be determined based on a graph adjacency matrix (e.g., in the context of a graph-based simulation problem).

In some embodiments, after act 820, the process 800 may proceed to act 830 in which interference between the first quantum state and the second quantum state may be caused by applying a beamsplitter interaction between the first quantum state and the second quantum state. Applying the beamsplitter interaction may be performed by applying an electromagnetic signal (e.g., a microwave signal) to a third ancilla qubit coupled between the first and second physical qubits. The beamsplitter interaction may cause interference between the first and second quantum states by causing four-wave mixing between the first and second quantum states.

In some embodiments, the electromagnetic signal having a frequency approximately equal to a difference between resonant frequencies of the first and second physical qubits. In some embodiments, the electromagnetic signal implementing the beamsplitter interaction may have parameters (e.g., beamsplitting parameters) determined based on a covariance matrix determined using a graph adjacency matrix (e.g., in the context of graph-based simulation problems).

In some embodiments, after act 840, the process 800 may proceed to act 850, in which the first quantum state stored in the first physical qubit may be measured. The first quantum state may be measured by applying to the first ancilla qubit two electromagnetic pulses having a frequency approximately equal to a resonant frequency of the first ancilla qubit followed by the application of an electromagnetic pulse having a frequency approximately equal to the resonant frequency of a readout resonator coupled to the first ancilla qubit, causing readout from the first physical qubit and measurement of mode M1 as depicted in FIG. 4B. The state of the first physical qubit may then be reset by applying an electromagnetic pulse to the coupling device.

In some embodiments, after act 850, the process 800 may proceed to act 860, in which a third quantum state may be initialized in the first physical qubit by applying a third squeezing operation to a vacuum state of the first physical qubit, as described in connection with FIG. 4C herein. In this manner, a large Gaussian state vector may be sampled by instantiating, interfering, and measuring quantum states using a compact quantum computing system.

In some embodiments, the compact quantum computing system may include a third physical qubit. In such embodiments, the process 800 may include additional acts of instantiating quantum states in the third physical qubit and causing interference between those states and the quantum states stored in the first and/or second physical qubit. For example, the process 800 may further include applying a third squeezing operation to a vacuum state of a third physical qubit to generate a fourth quantum state. The third squeezing operation may be implemented, for example, by applying an electromagnetic signal to a third ancilla qubit coupled to the third physical qubit (e.g., to cause three- or four-wave mixing between the third ancilla qubit and the third physical qubit). The electromagnetic signal may be, for example, a microwave signal having a frequency approximately equal to twice the resonant frequency of the third physical qubit. In some embodiments, parameters of the third squeezing operation may be determined based on a graph adjacency matrix (e.g., in the context of a graph-based simulation problem).

In some embodiments, after act 830 and before act 840, a second beamsplitter interaction may be applied between the second quantum state stored in the second physical qubit and the fourth quantum state stored in the third physical qubit. After the application of the second beamsplitter interaction, a third beamsplitter interaction may be applied between the first quantum state and the second quantum state.

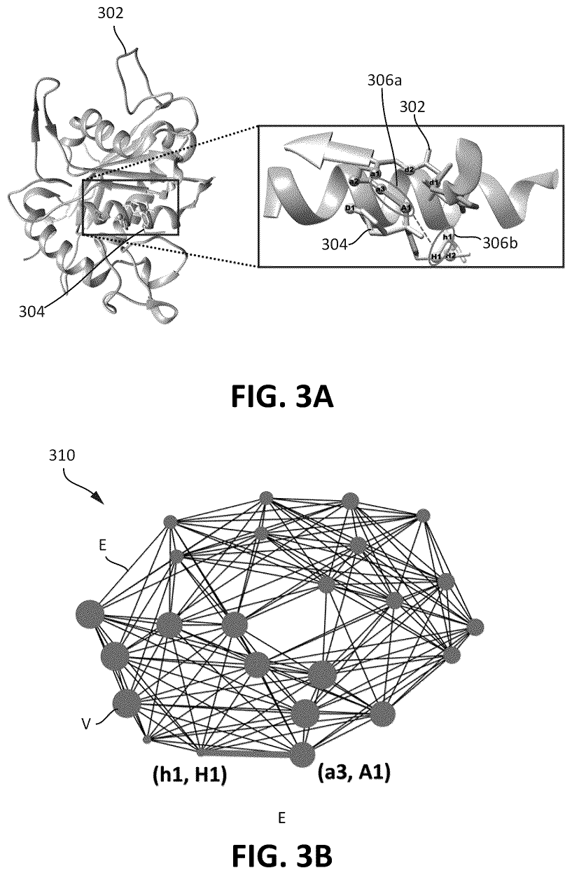

One example of a molecular docking problem is illustrated in FIG. 3A, in accordance with some embodiments of the technology described herein. In this example, the crystal structure 302 of the tumor necrosis factor-α converting enzyme receptor (TACE) and a thiol-containing aryl sulfonamide ligand 304 are depicted. In the inset of the image, potential binding interactions 306a and 306b are highlighted, showing two potential binding interactions between the enzyme receptor structure and the ligand structure that could occur concurrently. The binding interactions 306a and 306b involve four pharmacophores of the ligand (A1, D1, H1, and H2) and six pharmacophores of the enzyme receptor (a1, a2, a3, d1, d3, and h1).

The interactions between the crystal structure 302 and the ligand 304 may be encoded in a binding interaction graph 310, as shown in FIG. 3B, in accordance with some embodiments of the technology described herein. The binding interaction graph 310 shows all possible pairwise ligand-receptor interactions for the molecular docking problem of FIG. 3A, with vertices, V, corresponding to pairwise interactions and edges, E, linking interactions that can occur concurrently. Larger vertices indicate heavier weights (e.g., stronger interactions).

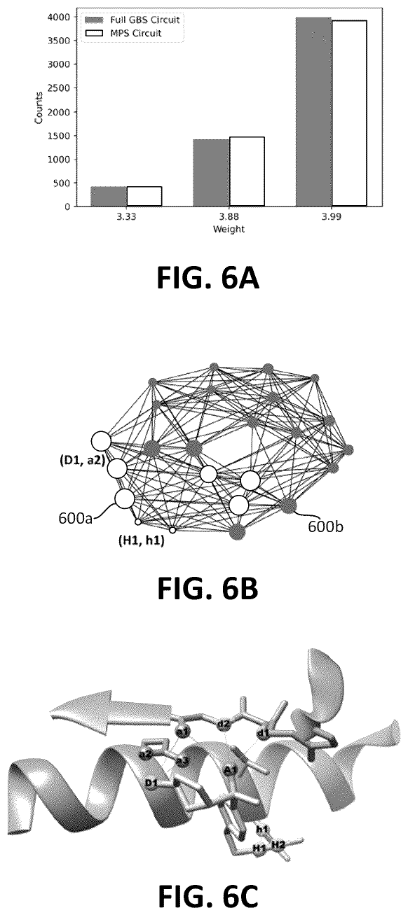

FIGS. 6A-6C illustrate solutions to the molecular docking problem of FIGS. 3A-3B, in accordance with some embodiments of the technology described herein. FIG. 6A is a bar chart comparing calculated weights of three cliques (e.g., subgraphs of the binding interaction graph 310 indicating possible concurrent bindings) identified for the structure of FIG. 3A. The weights were sampled using a full Gaussian boson sampling circuit (e.g., similar to the examples of FIGS. 4A and 5A) and using a holographic implementation (e.g., similar to the examples of FIGS. 4B and 5B). As seen in FIG. 6A, both techniques result in similar weighted values and identified the same cliques of concurrent binding interactions.

FIG. 6B illustrates the binding interaction graph of FIG. 3B with the subgraph for the heaviest clique (weight=3.99) obtained by the calculations of FIG. 6A, in accordance with some embodiments of the technology described herein. The subgraph is indicated by the open vertices 600a, while closed vertices 600b indicate pairwise interactions that are not part of the selected clique. FIG. 6C is an illustration of binding interactions associated with the identified clique represented by the subgraph of FIG. 6B.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, a controller, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

### Example

Graph theory aids in the modeling of molecules, chemical datasets, and reaction networks, as shown in recent studies. It enables easier calculations in diverse areas from cheminformatics to quantum chemistry, and polymer chemistry. Despite its utility, the complex nature of molecules and chemical databases often results in large graphs, challenging the capabilities of classical algorithms. For instance, several classes of problems require the computation of permanents, which is known to be a #P-hard, escalating to #P-complete for binary matrices. Such complexity places these computations beyond the reach of conventional methods. Near-term quantum computers that implement Boson Sampling (BS) offer a promising quantum solution. Here, the application of BS to simulations of molecular docking is explored, focusing on the possibility of sampling subgraphs describing the interactions between a molecule and a biological receptor using compact bosonic processors.

BS involves the sampling of photons dispersed through a passive N-mode linear interferometer on quantum calculations of permanents and determinants. In such experiments, the outcome distribution is determined by the permanent of the N×N matrix representing the transition probability amplitudes of the linear interferometer. As a result, BS enables the sampling of bosons from a distribution that would be challenging to simulate classically for large values of N, offering a promising approach to tackling problems previously considered intractable.

BS has been implemented on a diverse array of hardware platforms, offering versatility and potential in applications of chemical relevance. Notably, BS has been applied to compute the vibronic spectra of triatomic molecules using 3D circuit Quantum Electrodynamics (cQED) processors, and to simulate molecular docking on photonic devices. In fact, the initial BS sampling setup utilized optical photons. However, the efficient generation and detection of non-classical light states pose significant challenges. Here, the use of microwave photons is explored within the cQED architecture, offering a promising alternative technology with unique advantages for implementing BS.

The cQED architecture stands at the forefront of quantum technology, featuring superconducting qubits based on Josephson junctions that are strongly coupled to the modes of superconducting microwave cavities. This setup facilitates efficient information exchange and enables universal quantum control over quantum states. Moreover, it supports quantum non-demolition (QND) measurements of photon numbers within the microwave cavities of the superconducting circuits. The precision in photon counting afforded by this architecture enhances the feasibility of implementing BS as evidenced by previous studies.

Presented herein is an exploration of the possibility of implementing BS simulations of molecular docking using 3D cQED processors. Molecular docking, a computational technique for drug design, predicts the binding configuration and most favorable interactions of a molecule and its receptor. This task is difficult for classical computers due to the need to perform an exhaustive search of possible molecular configurations. BS, with its ability to sample from a distribution defined by the permanent of the interaction matrix, holds promise for significantly enhancing the efficiency of molecular docking simulations, offering a novel approach to overcoming the limitations of traditional computational methods.

Gaussian boson sampling (GBS), a version of BS based on multimode Gaussian states, is focused on herein. The possibility of simulating GBS using 3D cQED processors employing a holographic approach is explored. This method is inspired by the successful utilization of GBS in conjunction with 3D cQED processors for simulations of molecules.

The methodology begins by approximating the multimode state vectors as low-rank matrix product states (MPS, also called tensor-trains), followed by the variational parametrization of quantum circuits to represent these statevectors. The resulting circuits are executed holographically by re-purposing modes, with the term “holographic” reflecting the dimension reduction benefit from the re-purposing algorithm, in echo with holography that constructs 3D image from 2D snapshots. This allows for hardware efficiency, enabling simulations of a few tens of modes with as few as 2-3 microwave modes in the cQED devices.

Numerical simulations reveal that these cQED circuits could accurately simulate multimode Gaussian states, closely matching the benchmark Gaussian states of conventional GBS applied to molecular docking problems. Therefore, it is anticipated that these findings will pave the way for experimental investigations of compact 3D cQED devices into molecular docking and other subgraph isomorphism problems, highlighting the potential for conducting realistic simulations with numerous modes on modestly sized cQED devices.

### Methodology

### Molecular Docking by Gaussian Boson Sampling

Ligand-receptor interactions (FIG. 3A) are established by ligand and receptor pharmacophores, as described in the section titled “Interaction graph” herein. The interaction graph (FIG. 3B) describes the network of compatible pairs of interactions where the vertices correspond to pairs of interactions while the edges connect interactions that can be established simultaneously. The section titled “Maximum clique subgraph” herein outlines how the interaction graph is mapped into a GBS device to generate a multimode Gaussian state vector, with covariance parameterized by the adjacency matrix of the interaction graph.

Simulations represent the state vector in MPS format (FIGS. 4A and 5A) allowing for a holographic implementation (FIGS. 4B and 5B) using a cQED device. Sampling from that state vector distribution reveals subgraphs with maximum cliques (FIGS. 6A-6B) corresponding to subsets of interactions that can be established simultaneously (FIG. 6C). The section titled “Holographic implementation” outlines experimental strategies for holographic implementations of GBS based on MPS networks with one and two layers of two-mode beam-splitters, parameterized to approximate the full GBS network. The section titled “Bosonic cQED hardware setup for holographic GBS” describes cQED devices based on bosonic 3D cQED devices for simulations of holographic MPS networks.

### Interaction Graph

Molecular docking algorithms predict favorable binding configurations of ligands (or drug-like molecules) as determined by interactions established by pharmacophores in the ligand with complementary pharmacophores in the target macromolecule (receptor). Configurations are ranked in terms of docking scores that give a rough estimate of relative binding affinities based on molecular descriptors.

Reliable determination of docking scores for a series of ligands and favorable binding configurations is particularly valuable since it allows for rapid in silico screening of a large number of compounds. That process can quickly identify promising lead compounds for subsequent more accurate analysis and discard unsuitable ligands. In the simplest approach, both the ligand and the receptor are approximated as rigid bodies, although methods that account for the inherent flexibility of the ligand and the receptor are available. Favorable ligand orientations at the binding site can be revealed by using the isomorphous subgraph matching method, as implemented in the DOCK 4.0, FLOG, and SQ algorithms. In that formulation of the binding problem, the ligand-receptor interactions are represented by the so-called interaction graph G (V, E), an example of which is shown in FIG. 3B. Here, the vertices, V, represent ligand-receptor interactions established by pharmacophores, while the edges, E, link pairs of interactions that can occur concurrently since the drug pharamacophores involved in these interactions are at the same distance apart as the corresponding pharmacophores in the receptor.

The interaction graph G(V, E) is defined by its M×M adjacency matrix A, where M>1 is the total number of possible ligand-receptor interactions, with Aᵢⱼ=1 if the interactions i and j are compatible (if both interactions can be established simultaneously), and Aᵢⱼ=0, otherwise. To model the interaction strength between pharmacophores, every vertex was weighed according to the type of pharmacophores i and j, biasing the strength of the intermolecular interactions with a pre-parameterized potential. Having defined the adjacency, the problem of docking is reduced to the so-called maximum clique problem, which involves finding the largest fully connected subgraph within the graph, representing the largest set of compatible interactions as determined by the types of pharmacophore interactions and the inter-pharmacophore distances.

### Maximum Clique Subgraph

GBS addresses the maximum clique problem, as shown below, through preparation of a multimode Gaussian state vector whose covariance matrix is parameterized by the adjacency matrix of the ligand-receptor interaction graph. The state vector is generated from a vacuum state, using single mode squeezing and a multimode linear interferometer. For simplicity, it is assumed that the mode count significantly exceeds the average photon number, resulting in either a single photon or no photon detection (nⱼ∈{0,1}) in each mode j.

### Multimode Gaussian of Bosonic Modes

A Gaussian state of a system with M bosonic modes, designated as j, with creation and annihilation operators âⱼ^{†} and âⱼ, can be described by the density matrix

$$\hat{ρ} = {( 2 π )}^{- M} {\vert det ( σ ) \vert}^{- 1 / 2} {e}^{- \frac{1}{2} {( \hat{ζ} - d )}^{†} {σ}^{- 1} ( \hat{ξ} - d )} \qquad ( 1 )$$

where \hat{ξ}=(â₁, . . . , â_{M}, â₁^{†}, . . . , â_{M}^{†})^{T} is a 2M-component vector of operators. According to Eq. (1), the Gaussian state is defined completely by its first and second statistical moments, d and σ, respectively. The first moments

$${d}_{j} = Tr [ \hat{ρ} {\hat{ξ}}_{j} ] \qquad ( 2 )$$

describe the mode displacements, while the second moments

$${σ}_{ij} = \frac{1}{2} Tr [ \hat{ρ} \{ ( {\hat{ξ}}_{i} - {d}_{i} ) , {( {\hat{ξ}}_{j} - {d}_{j} )}^{†} \} ] \qquad ( 3 )$$

define the 2M×2M covariance matrix σ in terms of the anti-commutators

$$\{ ( {\hat{ξ}}_{i} - {d}_{i} ) , {( {\hat{ξ}}_{j} - {d}_{j} )}^{†} \} = ( {\hat{ξ}}_{i} - {d}_{i} ) , {( {\hat{ξ}}_{j} - {d}_{j} )}^{†} + {( {\hat{ξ}}_{j} - {d}_{j} )}^{†} ( {\hat{ξ}}_{i} - {d}_{i} ) \qquad ( 4 )$$

Measurements of the modes, when the system is described by the Gaussian state vector defined by Eq. (1), report whether a photon is detected or not in each mode, as follows: ñ=(n₁, n₂, . . . , n_{M})^{T}, where nⱼ∈{0,1} with j=1, . . . , M. In the realm of molecular docking, each mode j symbolizes a distinct interaction between a pharmacophore of the drug and a pharmacophore of the receptor. Detecting a photon in a particular mode suggests that the corresponding interaction is established, whereas the absence of photons signifies a disruption of that interaction.

### Probability Distribution

The probability distribution of outputs ñ, obtained by measuring the multimode Gaussian in the Fock basis, is defined, as follows

$$Pr ( \overline{n} ) = Tr [ \hat{ρ} \hat{n} ] \qquad ( 5 )$$

where \hat{n}=⊗ⱼ₌₁^{M}\hat{n}ⱼ is the tensor product of number state operators \hat{n}ⱼ=|nⱼ [symbol] [symbol] nⱼ| corresponding to the probability of observing n, bosons in output mode j. The right-hand side of Eq. (5) can be expanded by using the P-representation of operators, as follows (see the section titled “P-representation of the density operator”):

$$Tr [ {{⊗}^{M}}_{j = 1} \vert {n}_{j} 〉 〈 {n}_{j} \vert \hat{ρ} ] = \int {P}_{{⊗}_{j = 1}^{M} \vert {n}_{j} 〉 〈 {n}_{j} \vert} ( α ) Q ( α ) {d}^{2} α \qquad ( 6 )$$

$$where$$

$${P}_{{⊗}_{j = 1}^{M} \vert {n}_{j} 〉 〈 {n}_{j} \vert} ( α ) = {\prod }_{j = 1}^{M} \frac{{e}^{{\vert {α}_{j} \vert}^{2}}}{{n}_{j} !} \frac{{\partial }^{2 {n}_{j}}}{{\partial }^{{n}_{j}} {α}_{j} {\partial }^{{n}_{j}} {α}_{j}^{*}} {δ}^{2} {α}_{j} \qquad ( 7 )$$

Q(α), introduced by Eq. (6), is the Husimi function of the Gaussian state, defined as follows (see the section titled “P-representation of the density operator”):

$$Q ( α ) = {π}^{- M} 〈 α \vert \hat{ρ} \vert α 〉 = \frac{{π}^{- M}}{\sqrt{\vert det ( {σ}_{Q} ) \vert}} {e}^{- {α}^{†} {σ}_{Q}^{- 1} α} \qquad ( 8 )$$

where α=(α₁, . . . , α_{M}, α₁*, . . . , α_{M}*)^{T} and σ_{Q}=σ+I_{2M}/2, with I_{2M} being the 2M×2M identity matrix. The Husimi function obtained with Eq. (8), using the Gaussian density matrix introduced by Eq. (1), can be substituted into Eq. (6) to obtain (see the section titled “Output Probabilities of an M-mode Gaussian”)

$${Pr ( \overline{n} ) = \frac{1}{\sqrt{\vert det ( {σ}_{Q} ) \vert}} {\prod }_{j = 1}^{M} \frac{1}{{n}_{j} !} \frac{{\partial }^{2 {n}_{j}}}{{\partial }^{{n}_{j}} {α}_{j} {\partial }^{{n}_{j}} {α}_{j}^{*}} {e}^{\frac{1}{2} {α}^{T} K α} \vert}_{{α}_{j} = 0} \qquad ( 9 )$$

$$where$$

$$K = {X}_{2 M} ( {I}_{2 M} - {σ}_{Q}^{- 1} ) \qquad ( 10 )$$

with

$${X}_{2 M} = [ \begin{matrix}0 & {I}_{M} \\ {I}_{M} & 0\end{matrix} ] .$$

Since nⱼ∈{0,1}, the multivariate derivatives in Eq. (9) can be easily evaluated by using the high-order chain rule given by the Faà di Bruno's formula (see the section titled “Faà di Bruno's formula”), as follows:

$$Pr ( \overline{n} ) = \frac{1}{\overline{n} ! \sqrt{\vert det ( {σ}_{Q} ) \vert}} {\sum }_{ρ ∈ {P}_{2 N}^{2}} {\prod }_{\{ i , j \}} {K}_{ij} \qquad ( 11 )$$

where N=n₁+ . . . +n_{M} is the total number of modes where a photon was detected according to ñ, and ñ!=n₁!n₂! . . . n_{M}!. Moreover, P_{2N}² is the set of partitions of the set {1, 2, . . . , 2N} into subsets of two indices. The summation in Eq. (11) is exactly the Hafnian of the 2N×2N submatrix of K with rows and columns corresponding to the modes where photons were detected (i.e., modes j with nⱼ=1). Defining the submatrix of those modes as matrix K_{S}, the following is obtained,

$$Pr ( \overline{n} ) = \frac{1}{\overline{n} ! \sqrt{\vert det ( {σ}_{Q} ) \vert}} Haf ( {K}_{S} ) \qquad ( 12 )$$

To encode graph problems into Gaussian quantum states, the matrix K is considered to be defined in terms of the graph adjacency matrix A, as follows

$$K = c ( A ⊕ A ) \qquad ( 13 )$$

where the circled plus sign denotes the construction of a block-diagonal matrix from the component matrices. Furthermore, 0<c<λ₁⁻¹ is a positive rescaling parameter, with λ₁>0 the maximum eigenvalue of A. Note that for an undirected graph without self-connection, A is a real symmetric matrix with all diagonal elements being 0. Since Tr [A]=0 implies that the eigenvalues must be either all 0 or containing both positive and negative numbers, and the only real symmetric matrix that contains all zero eigenvalues is the zero matrix, the largest eigenvalue of A must be positive for a non-zero A. In particular, the scaling factor ensures that K corresponds to a valid covariance matrix σ as in Eq. (10), resulting in a probability distribution Pr (n̅) that is bounded between zero and one. In the section titled “Quantum Circuit” herein it is shown that an M-mode Gaussian state with such a K can always be prepared by a programmed optical network.

Substituting Eq. (13) into Eq. (12), the following is obtained

$$\begin{matrix}Pr ( \overline{n} ) = \frac{{c}^{N}}{\overline{n} ! \sqrt{\vert det ( {σ}_{Q} ) \vert}} Haf ( {A}_{S} ⊕ {A}_{S} ) \\ = \frac{{c}^{N}}{\overline{n} ! \sqrt{\vert det ( {σ}_{Q} ) \vert}} Haf {( {A}_{S} )}^{2}\end{matrix} \qquad ( 14 )$$

providing the probability distribution of outputs n in terms of the Hafnian of A_{S} (with A_{S} the submatrix of A defined by the intersection of rows and columns of A corresponding to the modes where photons were detected).

### Binary Graphs

In the simplest formulation of a binary graph, A is the M×M real and symmetric adjacency matrix with Aᵢⱼ∈{0,1}. Aᵢⱼ=1 denotes that nodes i and j share an edge, while Aᵢⱼ=0 denotes that nodes i and j are not connected. In the context of binding interaction graphs that are of primary interest, a node denotes a binding interaction between a pair of pharmacophore points, and connection between two nodes indicate the two binding interactions can be established simultaneously, while no connection indicates that the two binding interactions cannot be concurrently established because by establishing one interaction the other one is disrupted. This binary approach can be generalized to account for the strength of the connections, using instead a weighted binding interaction graph as described later in the section “Weighted Graphs” herein.

It is important to note that each term in the sum of the Hafnian in Eq. (14) represents a perfect matching where each node of the subgraph is connected to one (and only one) other node. As a result, the Hafnian gives the count of perfect matchings, and thus reveals the number of possible ways the drug pharmacophores can concurrently establish interactions with pharmacophores in the receptor. As per Eq. (14), subgraphs with larger Hafnian are sampled with higher probability.

Gaussian states are prepared with covariance σ, obtained by inverting Eq. (10), as follows

$$σ = {( {I}_{2 M} - {X}_{2 M} K )}^{- 1} - \frac{{I}_{2 M}}{2} \qquad ( 15 )$$

with K defined according to Eq. (13). This is achieved by using a quantum circuit that integrates single mode squeezing gates and a multimode linear interferometer, as detailed in the section titled “Quantum Circuit” herein. Sampling from the state vector generated by that circuit enable the effective identification of the subgraphs with larger Hafnian within the interaction graph, revealing the capability of the drug to establish concurrent interactions with the receptor.

### Weighted Graphs

Extending the method to accommodate weighted graphs, beyond binary adjacency matrices A (with Aᵢⱼ∈{0,1}) is straightforward. This uses a weighting vector Ω, where each element, Ωᵢₗ=c(1+wᵢ), is defined by a weight wᵢ, assigned to the i^{th} node. This design of weighting vector ensures that subgraphs with a larger weight are favored during sampling. In the context of molecular docking, these weights are obtained from a knowledge-based potential, where a heavier weight wᵢ corresponds to a stronger binding interaction. For instance, if the i^{th} node corresponds to a hydrogen bond donor-acceptor interaction, wᵢ would be larger than nodes representing weaker interactions (e.g., hydrophobic contacts). The weighting vector Ω is written as a diagonal matrix (diag (Ω)→Ω) and applied on both sides of A, to generate a weighted graph adjacency matrix, as follows

$$A ↦ Ω A Ω \qquad ( 16 )$$

generates a photon distribution defined by both A and Ω, as follows

$$\begin{matrix}Pr ( \overline{n} ) = {\frac{{c}^{N}}{\overline{n} ! \sqrt{\vert det ( {σ}_{Q} ) \vert}} [ Haf ( {Ω}_{S} {A}_{S} {Ω}_{S} ) ]}^{2} \\ = {\frac{{c}^{N}}{\overline{n} ! \sqrt{\vert det ( {σ}_{Q} ) \vert}} [ {\sum }_{ρ ∈ {P}_{N}^{2}} {\prod }_{\{ i , j \} ∈ ρ} {Ω}_{ii} {A}_{ij} {Ω}_{jj} ]}^{2} \\ = {\frac{{c}^{N}}{\overline{n} ! \sqrt{\vert det ( {σ}_{Q} ) \vert}} [ det ( Ω ) {\sum }_{ρ ∈ {P}_{N}^{2}} {\prod }_{\{ i , j \} ∈ ρ} {A}_{ij} ]}^{2} \\ = {\frac{{c}^{N}}{\overline{n} ! \sqrt{\vert det ( {σ}_{Q} ) \vert}} [ det ( {Ω}_{S} ) Haf ( {A}_{S} ) ]}^{2}\end{matrix} , \qquad ( 14 )$$

(17)
where Ω_{S} is the submatrix of Ω corresponding to the modes registering photons (the modes that also define A_{S}). According to Eq. (17), the resulting GBS with rescaled adjacency matrices has higher probability of sampling cliques with a large number of strongly interacting pharmacophores.

### Quantum Circuit

Here, it is shown how to build a quantum circuit of M modes that generates the desired multimode Gaussian state with covariance defined by Eq. (15) with K=c(A⊕A). In particular, it is shown that such a state can be obtained by passing a vacuum Gaussian state through an optical network composed of M single-mode squeezers and an M-mode interferometer, both parameterized according to A. Firstly, it is shown how the single-mode squeezers and M-mode interferometer transform the M-mode vacuum state by preserving its Gaussian shape but changing its covariance. Then, it is shown how the squeezers and interferometer are parameterized according to A.

The single mode vacuum state |0 [symbol] is the eigenstate of â with eigenvalue 0:

$$\hat{a} \vert 0 〉 = 0. \qquad ( 18 )$$

### Therefore,

$$\begin{matrix}{〈 \hat{a} 〉}_{vac} = Tr [ \vert 0 〉 〈 0 \vert \hat{a} ] = 0 \\ {〈 {\hat{a}}^{†} 〉}_{vac} = Tr [ \vert 0 〉 〈 0 \vert {\hat{a}}^{†} ] = 0 \\ {〈 \hat{a} \hat{a} 〉}_{vac} = Tr [ \vert 0 〉 〈 0 \vert \hat{a} \hat{a} ] = 0 \\ {〈 \hat{a} {\hat{a}}^{†} 〉}_{vac} = Tr [ \vert 0 〉 〈 0 \vert \hat{a} {\hat{a}}^{†} ] = 1 \\ {〈 {\hat{a}}^{†} \hat{a} 〉}_{vac} = Tr [ \vert 0 〉 〈 0 \vert {\hat{a}}^{†} \hat{a} ] = 0 \\ {〈 {\hat{a}}^{†} {\hat{a}}^{†} 〉}_{vac} = Tr [ \vert 0 〉 〈 0 \vert {\hat{a}}^{†} {\hat{a}}^{†} ] = 0\end{matrix} , \qquad ( 19 )$$

where the subscripts indicate that the expectation values [symbol] · [symbol] are evaluated with \hat{ρ}=|0 [symbol] [symbol] 0|. Substituting Eq. (19) into Eq. (3), the covariance matrix of the single mode vacuum state is obtained

$${σ}_{vac} = \frac{1}{2} [ \begin{matrix}1 & 0 \\ 0 & 1\end{matrix} ] . \qquad ( 20 )$$

The single mode squeezing operation is defined, as follows

$$\hat{S} ( r ) = {e}^{( - {r ( \hat{a} )}^{2} + {r ( {\hat{a}}^{†} )}^{2} ) / 2} \qquad ( 21 )$$

where r∈ [symbol] is real-valued and is referred to as the squeezing parameter. The section titled “Covariance matrix of squeezed and rotated state” (Eq. (116)) shows that in the Heisenberg picture, the action of the squeezing operation transforms the annihilation and creation operators, as follows

$$\begin{matrix}{\hat{a}}^{′} = {\hat{S} ( r )}^{†} \hat{a} \hat{S} ( r ) = cosh ( r ) \hat{a} + sinh ( r ) {\hat{a}}^{†} \\ {( {\hat{a}}^{′} )}^{†} = {\hat{S} ( r )}^{†} {\hat{a}}^{†} \hat{S} ( r ) = cosh ( r ) {\hat{a}}^{†} + sinh ( r ) \hat{a}\end{matrix} . \qquad ( 22 )$$

Substituting Eq. (22) into Eq. (3), it is found that the single mode squeezed state is again Gaussian with the following covariance matrix (Eq. (123))

$${σ}^{′} ( r ) = \frac{1}{2} [ \begin{matrix}{cosh}^{2} ( r ) + {sinh}^{2} ( r ) & 2 cosh ( r ) sinh ( r ) \\ 2 cosh ( r ) sinh ( r ) & {cosh}^{2} ( r ) + {sinh}^{2} ( r )\end{matrix} ] . \qquad ( 23 )$$

Generalizing to M>1 modes, the following is obtained

$${σ}_{sque} = \frac{1}{2} [ \begin{matrix}{⊕}_{j = 1}^{M} {cosh}^{2} ( {r}_{j} ) + {sinh}^{2} ( {r}_{j} ) & {⊕}_{j = 1}^{M} 2 cosh ( {r}_{j} ) sinh ( {r}_{j} ) \\ {⊕}_{j = 1}^{M} 2 cosh ( {r}_{j} ) sinh ( {r}_{j} ) & {⊕}_{j = 1}^{M} {cosh}^{2} ( {r}_{j} ) + {sinh}^{2} ( {r}_{j} )\end{matrix} ] . \qquad ( 24 )$$

Next, the effect of the M-mode interferometer on the squeezed state covariance matrix is evaluated by starting from the simplest 2-mode case and then obtaining the M-mode case.

The smallest interferometer is the 2-mode beam-splitter. It transforms a two-mode state according to the following operator

$$\hat{B} ( θ ) = {e}^{θ ( {\hat{a}}_{1}^{†} {\hat{a}}_{2} - {\hat{a}}_{1} {\hat{a}}_{2}^{†} )} , \qquad ( 25 )$$

where θ∈[0,2π] is a given angle. In the Heisenberg picture, \hat{B}(θ) transforms the annihilation operators, as follows

$${\hat{a}}_{1}^{′} = B {( θ )}^{†} {\hat{a}}_{1} B ( θ ) = cos ( θ ) {\hat{a}}_{1} + sin ( θ ) {\hat{a}}_{2} \qquad ( 26 )$$

$${\hat{a}}_{2}^{′} = B {( θ )}^{†} {\hat{a}}_{2} B ( θ ) = cos ( θ ) {\hat{a}}_{2} - sin ( θ ) {\hat{a}}_{1} .$$

Hence, for k=1, 2 the following is obtained

$${\hat{a}}_{k}^{′} = {\sum }_{j = 1}^{2} {U}_{kj} {\hat{a}}_{k} \qquad ( 27 )$$

$${( {\hat{a}}_{k}^{′} )}^{†} = {\sum }_{j = 1}^{2} {U}_{kj}^{*} {\hat{a}}_{k}^{†}$$

where

$$U = U ( θ ) = [ \begin{matrix}cos ( θ ) & sin ( θ ) \\ - sin ( θ ) & cos ( θ )\end{matrix} ]$$

is a unitary transformation. Additionally, when a phase-shifter is placed after the beam-splitter such that

$${\hat{a}}_{1} ↦ {e}^{i {ϕ}_{1}} {\hat{a}}_{1}$$

for ϕ₁∈[0,2π], the resulting unitary transformation becomes

$$U = U ( θ , {ϕ}_{1} ) = [ \begin{matrix}cos ( θ ) {e}^{i {ϕ}_{1}} & sin ( θ ) {e}^{i {ϕ}_{1}} \\ - sin ( θ ) & cos ( θ )\end{matrix} ] .$$

Note that any 2×2 unitary matrix U can be implemented like this using a beam-splitter and a phase-shifter with a suitable choice of values for θ and ϕ₁. The generalization to M modes is straightforward and involves an M-mode interferometer, as follows:

$${\hat{a}}_{k}^{′} = {\sum }_{j}^{M} {U}_{kj} {\hat{a}}_{j} \qquad ( 28 )$$

$${( {\hat{a}}_{k}^{′} )}^{†} = {\sum }_{j}^{M} {U}_{kj}^{*} {\hat{a}}_{j}^{†}$$

where U is now an M×M unitary matrix. Substituting Eq. (28) into Eq. (3), it is found that the M-mode interferometer U transforms an arbitrary covariance matrix \tilde{σ}, as follows (Eq. (128))

$${σ}_{rot} = [ \begin{matrix}U & 0 \\ 0 & {U}^{*}\end{matrix} ] {\tilde{σ} [ \begin{matrix}{U}^{*} & 0 \\ 0 & U\end{matrix} ]}^{T} \qquad ( 29 )$$

Therefore, the outgoing Gaussian state obtained by rotating an M-mode squeezed vacuum state is described by the following covariance matrix

$${σ}_{out} = [ \begin{matrix}U & 0 \\ 0 & {U}^{*}\end{matrix} ] {{σ}_{sque} [ \begin{matrix}{U}^{*} & 0 \\ 0 & U\end{matrix} ]}^{T} \qquad ( 30 )$$

With the general covariance matrix defined, an explanation is now provided on how the parameters of the squeezers and interferometer are obtained to ensure that gout corresponds to Eq. (15), with kernel K defined by Eq. (13)

According to Takagi's matrix factorization, the symmetric matrix A can be decomposed, as follows

$$A = U \sum {U}^{T} = U ( \frac{1}{c} {⊕}_{j = 1}^{M} tanh ( {r}_{j} ) ) {U}^{T} \qquad ( 31 )$$

where Σ is the diagonal matrix of singular values of A, and 0<c<λ₁⁻¹ is a rescaling parameter, with λ₁>0 the maximum eigenvalue of A. The rescaling ensures that every singular value can be represented as tanh(rⱼ)/c (note that |tanh(r)|<1 for all r∈ [symbol] ). The unitary matrix U is used to parameterize the M-mode interferometer. The resulting covariance defined by Eq. (30) is consistent with the kernel K, introduced by Eq. (13), as shown in the section titled “Kernel matrix for the N-mode rotated state” where Eq. (13) is obtained by substituting Eq. (30) into Eq. (10).

The section titled “Holographic implementation” introduces an efficient implementation of the multimode Gaussian, with hardware efficiency, by first squeezing each mode and then implementing U in MPS format by using an M-mode passive interferometer. Measurement of the modes generates a probability distribution of photon outcomes as defined by Eq. (17). It is noted that the theoretical analyses in this subsection neglects errors in the squeezed states, which could potentially hinder the performance of the quantum device.

### Holographic Implementation

FIGS. 4A-4B and 5A-5B illustrate the implementation of circuits corresponding to the state of a system of 4 modes, such as a multimode Gaussian state, in MPS format. Note that each beam-splitter (BS) couples two adjacent bosonic modes.

### MPS Circuits

FIG. 4A shows the MPS circuit with one layer of beam-splitters, inspired by a circuit previously proposed for moderately entangled quantum states. FIG. 1B shows a compact 3D cQED device that could implement the circuit with hardware efficiency using the so-called holographic quantum computing approach shown in FIG. 4B. Note that the circuits shown in FIGS. 4A and 4B are equivalent. The only difference is that the circuit in (b) re-purposes the modes after measurement. Mode 1(q₁) is measured after squeezing and coupling with mode 2 by implementing the gates S₁, S₂, and BS₁₂. After that measurement, mode 1 can be repurposed for representing mode 3(q₃) by first squeezing with S₃ and coupling it with mode 2 according to BS₂₃. Mode 2 is measured and then re-purposed as mode 4. After applying S₄, BS₃₄, mode 3 and 4 are measured completing the implementation of the circuit with 4 modes, using a 2-mode quantum device. Circuits with more modes could be implemented analogously with the 2-mode device by iterative squeezing, coupling and measuring modes. FIGS. 4A and 4B also show that the gate cost of the holographic algorithm is O(m) for an m-mode Gaussian state.

States with more entanglement can also be implemented with hardware efficiency by employing a few more modes. As an example, FIG. 5A shows a 4-mode circuit with a higher level of entanglement established by a second layer of beam-splitters. Here, the circuit was implemented by employing a 3-mode device. FIG. 5B shows the holographic implementation starting with gates S₁, S₂, S₃, BS₁₂, BS₂₃, BS₁₂′, that could be implemented with the device illustrated in FIG. 2. Mode 1 would be measured first and re-purposed for representing mode 4. After this resetting, gates S₄, BS₃₄, BS₂₃′, BS₃₄′ are implemented, and modes 2-4 are finally measured. The resulting sampling is equivalent to that of the conventional GBS experiment.

### Parametrization

This section provides an explanation of how the beamsplitters employed in the MPS circuits introduced in the section titled “MPS Circuits” were parameterized, including the quantum circuits shown in FIGS. 4A-4B and 5A-5B with one and two layers of beam-splitters. As discussed in the section titled “Results and Discussion,” the resulting parametrization allows for efficient and accurate GBS simulations based on arbitrary adjacency matrices.

First, the covariance matrix of the reference state σ_{ref} is obtained, as well as the single mode squeezing parameters from the adjacency matrix A, as described in the section titled “Quantum Circuit.” Next, the single-mode squeezed states were prepared from vacuum states and fed into the MPS-based network with variational beam-splitting and phase-shifting parameters (θ=(θ₁, . . . , θ_{K}), ϕ=(ϕ₁, . . . , ϕ_{K})) for a network with a total of K beam-splitters. These parameters are optimized by minimization of the Frobenius distance dₜₘₚ(θ, ϕ)=∥σₜₘₚ(θ, ϕ)−θ_{ref}∥_{F} using a standard conjugate gradient-descent optimizer, which has the computational complexity of O(dm⁴) for an m-mode covariance matrix, with d is the number of iterations. The parameter set θ_{fin}, ϕ_{fin} corresponding to the minimal distance is then used as parameters for the quantum circuits.

Bosonic cQED Hardware Setup for Holographic GBS

The experimental requirements for implementing the holographic GBS include a high coherence multi-mode cavity system (with at least 2 storage modes), pair-wise beam-splitter operations with programmable phases and splitting ratios, and efficient readout, reset, and squeezing of individual cavity states. For example, the two-mode device used to demonstrate bosonic two-qubit gates, as shown in FIG. 1B, can already be used to implement GBS of arbitrarily number of modes in principle, using the circuits shown in FIG. 4B. More recent demonstration of a quantum router connecting 4 storage modes provides tools to implement the holographic GBS routine with added beam-splitter layers such as those shown in FIG. 5B. In this section, the techniques to physically implement the desired bosonic operations are briefly reviewed, the limitations of existing hardware are discussed, and a minimal construction of 3D circuit QED device tailored for holographic GBS is introduced.

Simply speaking, the key figure of merit that characterizes the performance of a device hardware for the proposed GBS protocol is the ratio of cavity coherence times and the operational cycle time. This ratio sets an upper bound on the executable circuit depth or effective number of modes that can be computed. The current state of the art in multimode cavity QED has led to quantum control of over 10 cavity modes with millisecond lifetimes. The coaxial stub geometry, which is more widely adopted, consistently exhibits coherence times at the millisecond level. It also offers spatial separation of the modes, facilitating individual control. In principle, seamless designs of superconducting cavities, leveraging low surface-to-volume ratio and materials processing technologies borrowed from particle accelerators, can achieve coherence times on the order of seconds. This represents a significant potential for enhancing system performance by several orders-of-magnitude.

Programmable two-mode beam-splitter and single-mode squeezing gates can be achieved with either the four-wave mixing or the three-wave mixing process using external microwave pumps in 3D circuit QED. In either case, the external pump, applied under the appropriate frequency matching condition, can activate a rotating-frame Hamiltonian of the form â^{†}\hat{b}e^{iθ}^{ᵢ}+â\hat{b}^{†}e^{-iθ}^{ᵢ} for photon conversion between â and \hat{b} modes or â^{†}e^{iϕ}^{ᵢ}+â²e^{-iϕ}^{ᵢ} for squeezing drives in mode â for the i^{th} beamsplitter. The phase of the pump tone controls the beam-splitting phase θᵢ or the squeezing phase ϕᵢ, while the amplitude and length of the external pump controls the splitting ratio or squeezing ratio. The four-wave mixing process is ubiquitously available in standard circuit QED hardware systems today, requiring only a fixed-frequency transmon ancilla, which generally allows for gates on the order of several μs. Much faster gates on 100 ns scale or shorter can be implemented with parametric charge or flux drives using novel Josephson ancilla circuitry that supports three-wave mixing processes. These ancillae including, for example, SNAIL (Superconducting Nonlinear Asymmetric Inductive eLement), RF SQUID, and ATS have been under intensive development recently. Their integration with high-coherence 3D systems has led to record beam-splitter fidelity in the range of 99.9%-99.99%.

Measurement and reset of cavities can use transmons ancillae and their low-Q readout resonators. A transmon qubit dispersively coupled to both a storage cavity and a readout resonator has been a well-developed tool to analyze the storage photon number using Ramsey sequences or selective transmon excitations. If the task is to distinguish between |0 [symbol] and |1 [symbol] for the cavity, a Ramsey-like protocol for photon-number parity measurement is preferable for its faster speed, which should allow cavity measurements within 1 μs for typical coupling parameters. Cavity-state reset for arbitrary initial photon number can employ a beamsplitter between the cavity and a linear low-Q mode (such as the readout resonators in FIG. 1B. To achieve a reasonable operation speed (μs or less), however, a dedicated parametric coupler capable of strong 3-wave mixing (e.g. a SNAIL or other suitable parametric coupler) is preferred. Assuming the expected number of cavity photons is low, an alternative approach involves resetting specific number states to vacuum through intermediary transmon states. Specifically, it is possible to convert a single-photon excitation in the storage cavity into double excitations in the transmon using a four-wave mixing drive. Subsequently, resetting the transmon should allow the resetting of the cavity |1 [symbol] state within 1 μs.

In constructing a full device capable of the proposed holographic GBS, a key consideration is the ability to measure and reset one cavity mode without affecting the other cavity modes. Therefore, a transmon ancilla with simultaneous dispersive coupling to multiple storage modes for their readout, is undesirable. Instead of designing spatially separated of storage cavity modes with independent transmon ancillas, a hardware-efficient setup is proposed that satisfies all the requirements as in FIG. 2. The storage cavity module containing multiple modes is coupled to a 3-wave mixing coupler (e.g. SNAIL) operated under the condition to minimize the 4th order nonlinearity of the storage modes (self-Kerr and cross Kerr). The 3-wave coupler is further coupled to another high-Q transfer cavity with a transmon ancilla. The 3-wave coupler allows fast beam-splitter operations between storage modes as well as swapping between any storage mode and the transfer cavity (typically prepared in vacuum) for readout and initialization. The number of photons in the transfer cavity can be analyzed by leveraging the dispersive interaction with the transmon. Additionally, the reset of the transfer cavity can be carried out using transmon-mediated sideband drives, which are conditioned on the cavity states. Utilizing the transfer cavity not only protects the storage modes from spurious fourth-order nonlinearities but also protects the low-Q components of the circuits, which provide fast measurement and reset speeds.

### Results and Discussion

Demonstrated herein are the capabilities of the MPS-based holographic approach as applied to GBS for solving the problem of molecular docking (see the section titled “Molecular docking mapped as a graph search problem”). Next, the scalability of the approach as applied to random adjacency matrices is analyzed (see the section titled “Scalability analysis with random graphs”).

### Molecular Docking Mapped as a Graph Search Problem

In this section, the MPS-based holographic approach is applied to solve the molecular docking problem which involves finding the optimal binding mode of a small drug molecule bound to a target biological receptor. Specifically, the benchmark model system of a thiol-containing aryl sulfonamide compound (AS) bound to the tumor necrosis factor-α converting enzyme (TACE), shown in FIG. 3A, that allows for direct comparisons of the MPS-based GBS approach to full GBS simulations is focused on. Binding of AS to TACE is determined by hydrogen-bonds and hydrophobic contacts established by 6 pharmacophores in the TACE active site and 4 pharmacophores in the AS. Therefore, there is a total of 24 possible pairs of interaction that could be established upon AS binding.

The binding interaction graph is thus defined by a 24×24 adjacency matrix A where Aᵢⱼ=Aⱼᵢ=1, if the two binding interactions i and j are geometrically compatible, and Aᵢⱼ=0, otherwise. With this adjacency matrix, the GBS routine is carried out as described in the section titled “Molecular docking by Gaussian boson sampling,” and the corresponding MPS-based holographic implementation is carried out as described in the section titled “Holographic implementation.” The results of the sampling are analyzed to identify the densest sampled subgraph.

FIGS. 6A-6C show the result of GBS versus MPS-network sampling. Dense subgraphs sampled by GBS are converted to heaviest cliques representing the most probable binding patterns, using an algorithm for post-processing GBS data. The algorithm first shrinks the GBS sampled subgraph into smaller cliques by sequentially removing vertices with small degree, until finding a clique. Then, the found clique is locally expanded into a large clique according to a local search algorithm that expand the clique by one vertex that is fully connected with all vertices in the original clique. As shown in FIG. 6A, the sampling routines successfully identify the heaviest clique (weight=3.99) with high probability. Moreover, sampling results from the MPS network agree closely with the GBS result, confirming the accuracy of the proposed MPS-based holographic approach. FIG. 6B plots the maximum clique as a subgraph in the binding interaction graph, while FIG. 6C visualizes the binding interactions that correspond to the maximum clique.

Scalability Analysis with Random Graphs

Here, the scalability and accuracy of the MPS-based algorithm described in the section titled “Holographic implementation” is examined for a series of random graphs, specified by randomly generated adjacency matrices A that satisfy the conditions described at the beginning of the section titled “Molecular docking by Gaussian boson sampling.”

First, the capabilities of the variational approach is examined to approximate the full optical network by comparing the covariance matrix at the output of the optimized MPS-based holographic network to the reference covariance matrix generated by the conventional GBS setup (Table 1).

The results displayed in Table 1 indicate that MPS-circuits equipped with either one or two layers of beam-splitters achieve high precision in approximating the GBS output states, showing that the covariance matrix error remains below 5 percent, even for circuits comprising up to 50 modes.

TABLE 1
Performance of the MPS-based circuits parameterized by
the variational covariance matrix optimization. The number of
modes for each test is denoted by M. The relative distance between
covariance matrices is defined as ||σₒₚₜ − σ_{ref} ||/||σ_{ref} ||.

| | |
|---|---|
| (a) 1-layer MPS network | (b) 2-layers MPS network |

| M | Distance | M | Distance |
|---|---|---|---|
| 12 | 0.05 | 12 | 0.04 |
| 16 | 0.04 | 16 | 0.04 |
| 20 | 0.04 | 20 | 0.03 |
| 24 | 0.03 | 24 | 0.02 |
| 50 | 0.02 | 50 | 0.01 |

### Conclusion

Presented herein are quantum circuits designed for simulating multi-mode state vectors on 3D cQED processors, leveraging matrix product state representations. These circuits have been showcased through simulations of molecular docking, specifically focusing on the binding of a thiol-containing aryl sulfonamide ligand to the tumor necrosis factor-α converting enzyme receptor, utilizing holographic Gaussian boson sampling. These findings reveal the proposed MPS scheme based on cQED devices with only 2 or 3 modes is able to prepare multimode Gaussian states that closely approximate those obtained by the conventional GBS method even for systems with up to 50 modes. This approach opens the door to a broad spectrum of GBS applications that could be efficiently executed on compact 3D cQED processors using the holographic method.

### Representations of the Gaussian State

The goal of this section is to first introduce the eigenstates of the annihilation operator, the so-called coherent states |α [symbol] that fulfill the eigenvalue equation

$$\hat{a} \vert α 〉 = α \vert α 〉 \qquad ( 32 )$$

where α∈ [symbol] is a given complex number. Based on the coherent states, the so-called P-representation of density operators that will be used to unfold GBS will be introduced. It will be seen that a coherent state has a precise phase defined by the complex amplitude α, although an indefinite number of photons, like the state of coherent light in a laser beam. In contrast, a Fock state is an eigenstate of the number operator, corresponding to a fixed well-defined number of photons although completely arbitrary (random) phase. In the following, harmonic oscillator coherent states are focused on, while noting that generalization to anharmonic coherent states is readily available.

### Overview of the Coherent States

The goal of this subsection is to provide an overview of properties of coherent states that are useful for applications to the GBS.

### Displacement Operator

To begin, coherent states can be created, shown as follows

$$\hat{D} ( α ) \vert 0 〉 = \vert α 〉 \qquad ( 33 )$$

where |0 [symbol] is the vacuum state defined as the ground state of the harmonic oscillator, and \hat{D}(α) is the displacement operator, defined as follows

$$\hat{D} ( α ) = {e}^{α {\hat{a}}^{†} - {α}^{*} \hat{a}} \qquad ( 34 )$$

$$= {e}^{{\hat{a}}^{†}} {e}^{- {α}^{*} \hat{a}} {e}^{- \frac{1}{2} {\vert α \vert}^{2}}$$

The second row of Eq. (34) is obtained from the first one by using the Hausdorff formula

$${e}^{\hat{A} + \hat{B}} = {e}^{\hat{A}} {e}^{\hat{B}} {e}^{- \frac{1}{2} [ \hat{A} , \hat{B} ]} ,$$

with Â=αâ^{†} and \hat{B}=−α*â, which is valid if [Â, [Â, \hat{B}]]=[\hat{B}, [Â, \hat{B}]]=0, as in this case. Note that [Â, \hat{B}]=−|α|²[â^{†}, â]=|α|², since [â^{†}, â]=−1.

Eqs. (32) and (33) will be obtained by showing that, according to the Baker-Campbell-Hausdorff relation, \hat{D}(α)^{†}â\hat{D}(α)=â+α (part 1). Hence, with â|0 [symbol] =0, it can be concluded that \hat{D}(α)^{†}+â\hat{D}(α)|0 [symbol] =α|0 [symbol] , and â\hat{D}(α)|0 [symbol] =α\hat{D}(α)|0 [symbol] (part 2). Consequently, by definition, \hat{D}(α)|0 [symbol] is equal to |α [symbol] : the eigenfunction of â with eigenvalue α.

Beginning with part 1: Firstly, it is shown that \hat{D}(α)⁻¹=\hat{D}(−α)=\hat{D}(α)^{†}:

$$\hat{D} {( α )}^{- 1} = {e}^{\frac{1}{2} {\vert α \vert}^{2}} {e}^{{α}^{*} \hat{a}} {e}^{- α {\hat{a}}^{†}} \qquad ( 35 )$$

$$= {e}^{- \frac{1}{2} {\vert α \vert}^{2}} {e}^{- α {\hat{a}}^{†}} {e}^{{α}^{*} \hat{a}} = \hat{D} ( - α ) ,$$

where the first equality follows from \hat{D}(α)⁻¹\hat{D}(α)=1. The second row of Eq. (35) is obtained from the first one since

$${e}^{{α}^{*} \hat{a}} {e}^{- α {\hat{a}}^{†}} = {e}^{- α {\hat{a}}^{†}} {e}^{{α}^{*} \hat{a}} {e}^{- {\vert α \vert}^{2}} . \qquad ( 36 )$$

The Baker-Campbell-Hausdorff relation

$${e}^{\hat{A}} \hat{B} {e}^{- \hat{A}} = \hat{B} + [ \hat{A} , \hat{B} ] + \frac{1}{2} [ \hat{A} , [ \hat{A} , \hat{B} ] ] + ⋯ \qquad ( 37 )$$

can be used with Â=−αâ^{†}+α*â, and \hat{B}=â to show that

$${\hat{D} ( α )}^{†} \hat{a} \hat{D} ( α ) = \hat{a} + α , \qquad ( 38 )$$

since [Â, \hat{B}]=[−αâ^{†}+α*â, â]=α, and therefore [Â, [Â, \hat{B}]]=0. For part 2, the vacuum state |0 [symbol] is applied to Eq. (38) and obtain

$${\hat{D} ( α )}^{†} \hat{a} \hat{D} ( α ) \vert 0 〉 = α \vert 0 〉 , \qquad ( 39 )$$

since â |0 [symbol] =0. Therefore, according to Eq. (32), it is concluded that

$$\hat{D} ( α ) \vert 0 〉 = \vert α 〉 , \qquad ( 40 )$$

which indeed is Eq. (33).

### Series Expansion.

Substituting Eq. (34) into Eq. (40), the following is obtained

$$\vert α 〉 = {e}^{- \frac{1}{2} {\vert α \vert}^{2}} {e}^{a {\hat{a}}^{†}} {e}^{- {α}^{*} \hat{a}} \vert 0 〉 \qquad ( 41 )$$

and by expanding the exponentials in Taylor series, the following is obtained

$$\begin{matrix}\vert α 〉 = {e}^{- \frac{1}{2} {\vert α \vert}^{2}} {e}^{α {\hat{a}}^{†}} \vert 0 〉 \\ = {e}^{- \frac{1}{2} {\vert α \vert}^{2}} {\sum }_{n = 0}^{∞} \frac{{α}^{n}}{n !} {( {\hat{a}}^{†} )}^{n} \vert 0 〉 \\ = {e}^{- \frac{1}{2} {\vert α \vert}^{2}} {\sum }_{n = 0}^{∞} \frac{{α}^{n}}{\sqrt{n !}} \vert n 〉 ,\end{matrix} \qquad ( 42 )$$

where the third row is obtained by â^{†}|n [symbol] =\sqrt{n+1}|n+1 [symbol] . In particular, the following eigenvalue equation â|α [symbol] =α|α [symbol] is obtained since

$$\begin{matrix}\hat{a} \vert α 〉 = {e}^{- \frac{1}{2} {\vert α \vert}^{2}} {\sum }_{n = 1}^{∞} \frac{{α}^{n}}{\sqrt{n !}} \sqrt{n} \vert n - 1 〉 \\ = {e}^{- \frac{1}{2} {\vert α \vert}^{2}} {\sum }_{n = 1}^{∞} \frac{{α}^{n - 1}}{\sqrt{( n - 1 ) !}} \vert n - 1 〉 \\ = α {e}^{- \frac{1}{2} {\vert α \vert}^{2}} {\sum }_{n = 0}^{∞} \frac{{α}^{n}}{\sqrt{( n ) !}} \vert n 〉 .\end{matrix} \qquad ( 43 )$$

Given that the coherent state is an eigenstate of the annihilation operator, its complex conjugate is an eigenstate of the creation operator

$${{( \hat{a} \vert α 〉 )}^{†} = ( α \vert α 〉 )}^{†} \qquad ( 44 )$$

$$〈 α \vert {\hat{a}}^{†} = 〈 α \vert {α}^{⋆} . \qquad ( 45 )$$

### Overlap.

Coherent states are not orthogonal since, according to Eq. (42),

$$\begin{matrix}〈 β \vert α 〉 = {e}^{- \frac{1}{2} {\vert α \vert}^{2}} {e}^{- \frac{1}{2} {\vert β \vert}^{2}} {\sum }_{n = 0}^{∞} {\sum }_{m = 0}^{∞} \frac{{( {β}^{*} )}^{m} {α}^{n}}{\sqrt{m ! n !}} 〈 m \vert n 〉 \\ = {e}^{- \frac{1}{2} {\vert α \vert}^{2}} {e}^{- \frac{1}{2} {\vert β \vert}^{2}} {\sum }_{n = 0}^{∞} \frac{{( {β}^{*} )}^{n} {α}^{n}}{n !} \\ = {e}^{- \frac{1}{2} {\vert α \vert}^{2}} {e}^{- \frac{1}{2} {\vert β \vert}^{2}} {e}^{{β}^{*} α} \\ = {e}^{- \frac{1}{2} {\vert β - α \vert}^{2}} {e}^{\frac{1}{2} ( {β}^{*} α - {βα}^{*} )}\end{matrix} \qquad ( 46 )$$

### Expectation Values.

The expectation value of position,

$$\hat{x} = \sqrt{\frac{ℏ}{2 m ω}} ( a + {a}^{†} ) \qquad ( 47 )$$

follows from

$$〈 α \vert \hat{x} \vert α 〉 = 〈 α \vert \sqrt{\frac{ℏ}{2 m ω}} ( a + {a}^{†} ) \vert α 〉 \qquad ( 48 )$$

$$= \sqrt{\frac{ℏ}{2 m ω}} ( 〈 α \vert a \vert α 〉 + 〈 α \vert {a}^{†} \vert α 〉 ) \qquad ( 49 )$$

$$= \sqrt{\frac{ℏ}{2 m ω}} ( α 〈 α \vert α 〉 + {α}^{⋆} 〈 α \vert α 〉 ) \qquad ( 50 )$$

$$= \sqrt{\frac{ℏ}{2 m ω}} ( α + {α}^{⋆} ) \qquad ( 51 )$$

$$= \sqrt{\frac{2 ℏ}{m ω}} Re ( α ) \qquad ( 52 )$$

Likewise, the expectation value of the momentum,

$$\hat{p} = - i \sqrt{\frac{m ℏ ω}{2}} ( a - {a}^{†} ) \qquad ( 53 )$$

follows from

$$〈 α \vert \hat{p} \vert α 〉 = 〈 α \vert - i \sqrt{\frac{m ℏ ω}{2}} ( a - {a}^{†} ) \vert α 〉 \qquad ( 54 )$$

$$= - i \sqrt{\frac{m ℏ ω}{2}} ( 〈 α \vert a \vert α 〉 - 〈 α \vert {a}^{†} \vert α 〉 ) \qquad ( 55 )$$

$$= - i \sqrt{\frac{m ℏ ω}{2}} ( α 〈 α \vert α 〉 - {α}^{⋆} 〈 α \vert α 〉 ) \qquad ( 56 )$$

$$= - i \sqrt{\frac{m ℏ ω}{2}} ( α - {α}^{⋆} ) \qquad ( 57 )$$

$$= \sqrt{2 m ℏ ω} Im ( α ) \qquad ( 58 )$$

Therefore, according to Eq. (48) and Eq. (54), the following is obtained

$$α = {α}_{r} + i {α}_{i} = \sqrt{\frac{m ω}{2 ℏ}} {q}_{α} + i \frac{1}{\sqrt{2 m ℏ ω}} {p}_{α} \qquad ( 59 )$$

$$where$$

$${q}_{α} = 〈 α \vert \hat{x} \vert α 〉$$

$$and$$

$${p}_{α} = 〈 α \vert \hat{p} \vert α 〉$$

### Representation as Wavefunction.

The wavefunctions can be obtain by substituting the eigenfunctions of the Harmonic oscillator,

$$〈 x \vert n 〉 = {( {2}^{n} n ! )}^{- 1 / 2} {( \frac{m ω}{π ℏ} )}^{1 / 4} exp ( - {\tilde{x}}^{2} / 2 ) {H}_{n} ( \tilde{x} ) \qquad ( 60 )$$

into Eq. (42), where \tilde{x}=\sqrt{mω/ℏ}, with Hₙ the n th-Hermite polynomial, giving

$$〈 x \vert α 〉 = {( \frac{m ω}{π ℏ} )}^{1 / 4} {e}^{- \frac{1}{2} {\vert α \vert}^{2}} {e}^{- \frac{1}{2} {\tilde{x}}^{2}} {\sum }_{n = 0}^{∞} \frac{{( α / \sqrt{2} )}^{n}}{n !} {H}_{n} ( \tilde{x} ) \qquad ( 61 )$$

The vacuum state corresponding to n=0 photons is described by the wavefunction [symbol] x|0 [symbol] . According to Eq. (61), [symbol] x|0 [symbol] is also a coherent state with α=0 (i.e., the ground state of a harmonic oscillator with mass m and frequency ω). It is noted that the Hermite polynomials are given by the characteristic function

$${e}^{2 \tilde{x} t - {t}^{2}} = {\sum }_{n = 0}^{∞} {H}_{n} ( \tilde{x} ) \frac{{t}^{n}}{n !} \qquad ( 62 )$$

as can be verified by the Taylor expansion at \tilde{x}. Therefore, substituting the characteristic function into Eq. (61), with t=α/\sqrt{2}, the following is obtained

$$\begin{matrix}〈 x \vert α 〉 = {( \frac{m ω}{π ℏ} )}^{1 / 4} {e}^{- \frac{1}{2} {\vert α \vert}^{2}} {e}^{\frac{1}{2} {\tilde{x}}^{2}} {e}^{- {( \tilde{x} - α / \sqrt{2} )}^{2}} \\ = {( \frac{m ω}{π ℏ} )}^{1 / 4} {e}^{- \frac{1}{2} ( {α}_{r}^{2} + {α}_{i}^{2} )} {e}^{\frac{1}{2} {\tilde{x}}^{2}} {e}^{- {( \tilde{x} - {α}_{r} / \sqrt{2} - i {α}_{i} / \sqrt{2} )}^{2}} \\ = {( \frac{m ω}{π ℏ} )}^{1 / 4} {e}^{- \frac{1}{2} ( {α}_{r}^{2} + {α}_{i}^{2} )} {e}^{\frac{1}{2} {\tilde{x}}^{2}} {e}^{- ( {\tilde{x}}^{2} + {α}_{r}^{2} / 2 - {α}_{i}^{2} / 2 + i {α}_{r} {α}_{i} - \sqrt{2} \tilde{x} ( {α}_{r} + i {α}_{i} ) )} \\ = {( \frac{m ω}{π ℏ} )}^{1 / 4} {e}^{- {α}_{r}^{2}} {e}^{- \frac{1}{2} {\tilde{x}}^{2}} {e}^{\sqrt{2} \tilde{x} {α}_{r}} {e}^{- i {α}_{i} ( {α}_{r} - \sqrt{2} \tilde{x} )} \\ = {( \frac{m ω}{π ℏ} )}^{1 / 4} {e}^{- {( {α}_{r} - \tilde{x} / \sqrt{2} )}^{2}} {e}^{- i {α}_{i} ( {α}_{r} - \sqrt{2} \tilde{x} )}\end{matrix} \qquad ( 63 )$$

Substituting αᵣ and αᵢ in terms of q_{α} and p_{α}, the following is obtained

$$\begin{matrix}〈 x \vert α 〉 = {( \frac{m ω}{π ℏ} )}^{1 / 4} {e}^{- {( {q}_{α} \sqrt{\frac{m ω}{2 ℏ}} - x \sqrt{\frac{m ω}{2 ℏ}} )}^{2}} {e}^{{- {ip}_{α} \sqrt{\frac{1}{2 m ℏ ω}} ( {q}_{α} \sqrt{\frac{m ω}{2 ℏ}} - \sqrt{2} x \sqrt{\frac{m ω}{ℏ}} )}^{}} \\ = {( \frac{m ω}{π ℏ} )}^{1 / 4} {e}^{- ( \frac{m ω}{2 ℏ} ) {( x - {q}_{α} )}^{2}} {e}^{\frac{i}{ℏ} {p}_{α} ( x - {q}_{α} )} {e}^{\frac{i}{2 ℏ} {p}_{α} {q}_{α}} \\ = {( \frac{γ}{π} )}^{1 / 4} {e}^{- \frac{γ}{2} {( x - {q}_{α} )}^{2}} {e}^{\frac{i}{ℏ} {p}_{α} ( x - {q}_{α} )} {e}^{\frac{1}{2 ℏ} {p}_{α} {q}_{α}}\end{matrix} \qquad ( 64 )$$

$$with$$

$$γ = m ω / ℏ .$$

### P-Representation of the Density Operator

The Glauber-Sudarshan P-representation of the density operator, \hat{ρ} (i.e., the P-function, P(α)), is a pseudo-probability distribution defined, as follows

$$\hat{ρ} = \int P ( α ) \vert α 〉 〈 α \vert {d}^{2} α \qquad ( 65 )$$

where d²α=dRe (α) dIm (α). As shown below,

$$P ( α ) = \frac{{e}^{{\vert α \vert}^{2}}}{{π}^{2}} \int {e}^{- {α}^{*} u + {u}^{*} α} 〈 - u \vert \hat{ρ} \vert u 〉 {e}^{{\vert u \vert}^{2}} {d}^{2} u \qquad ( 66 )$$

To prove Eq. (66), [symbol] −u|\hat{ρ}|u [symbol] is computed by using Eq. (65),

$$〈 - u \vert \hat{ρ} \vert u 〉 = \int P ( α ) 〈 - u \vert α 〉 〈 α \vert u 〉 {d}^{2} α \qquad ( 67 )$$

Substituting [symbol] α|u [symbol] according to Eq. (46), the following is obtained

$$〈 - u \vert \hat{ρ} \vert u 〉 = \int P ( α ) {e}^{- \frac{1}{2} {\vert u \vert}^{2} - \frac{1}{2} {\vert α \vert}^{2} - {u}^{*} α} {e}^{- \frac{1}{2} {\vert u \vert}^{2} - \frac{1}{2} {\vert α \vert}^{2} + {α}^{*} u} {d}^{2} α = {e}^{- {\vert u \vert}^{2}} \int P ( α ) {e}^{- {\vert α \vert}^{2}} {e}^{{α}^{*} u - {u}^{*} α} {d}^{2} α \qquad ( 68 )$$

Introducing the variable substitution α=x+iy and u=x′+iy′, the following is obtained

$$〈 - u ( {x}^{′} , {y}^{′} ) \vert \hat{ρ} \vert u ( {x}^{′} , {y}^{′} ) 〉 {e}^{{\vert u ( {x}^{′} , {y}^{′} ) \vert}^{2}} = \int P ( α ( x , y ) ) {e}^{- {x}^{2} - {y}^{2}} {e}^{( x - i y ) ( {x}^{′} + i {y}^{′} ) - ( {x}^{′} - i {y}^{′} ) ( x + iy )} dx dy = \int P ( α ( x , y ) ) {e}^{- {x}^{2} - {y}^{2}} {e}^{- i 2 y {x}^{′} + i 2 {y}^{′} x} dx dy \qquad ( 69 )$$

Introducing the function I(\tilde{x}, \tilde{y}) by

$$\begin{matrix}I ( \tilde{x} , \tilde{y} ) = \frac{1}{{π}^{2}} \int {e}^{i 2 {x}^{′} \tilde{y} - i 2 {y}^{′} \tilde{x}} 〈 - u ( {x}^{′} , {y}^{′} ) \vert \hat{ρ} \vert u ( {x}^{′} , {y}^{′} ) 〉 {e}^{{\vert u ( {x}^{′} , {y}^{′} ) \vert}^{2}} {dx}^{′} {dy}^{′} \\ = \frac{1}{{π}^{2}} \int P ( α ( x , y ) ) {e}^{- {x}^{2} - {y}^{2}} \int {e}^{- i 2 ( y - \tilde{y} ) {x}^{′} + i {y}^{′} 2 ( x - \tilde{x} )} {dx}^{′} {dy}^{′} dx dy \\ = \frac{1}{{( 2 π )}^{2}} \int P ( α ( x , y ) ) {e}^{- {x}^{2} - {y}^{2}} \int {e}^{- i ( y - \tilde{y} ) {x}^{″} + i {y}^{″} ( x - \tilde{x} )} {dx}^{″} {dy}^{″} dx dy \\ = \int P ( α ( x , y ) ) {e}^{- {x}^{2} - {y}^{2}} δ ( y - \tilde{y} ) δ ( x - \tilde{x} ) dx dy\end{matrix} \qquad ( 70 )$$

it is concluded that

$$I ( \tilde{x} , \tilde{y} ) = P ( α ( \tilde{x} , \tilde{y} ) ) {e}^{- {\tilde{x}}^{2} - {\tilde{y}}^{2}} \qquad ( 71 )$$

which provides

$$P ( α ( x , y ) ) = \frac{{e}^{{x}^{2} + {y}^{2}}}{{π}^{2}} \int {e}^{i {x}^{′} y - i {y}^{′} x} 〈 - u ( {x}^{′} , {y}^{′} ) \vert \hat{ρ} \vert u ( {x}^{′} , {y}^{′} ) 〉 {e}^{{\vert u ( {x}^{′} , {y}^{′} ) \vert}^{2}} {dx}^{′} {dy}^{′} \qquad ( 72 )$$

$$P ( α ) = \frac{{e}^{{\vert α \vert}^{2}}}{{π}^{2}} \int {e}^{- {α}^{*} u + {u}^{*} α} 〈 - u \vert \hat{ρ} \vert u 〉 {e}^{{\vert u \vert}^{2}} {d}^{2} u$$

### Pure Coherent States.

For the pure state \hat{ρ}=|β [symbol] [symbol] β|, according to Eq. (46), the following is obtained

$$\begin{matrix}〈 - u \vert \hat{ρ} \vert u 〉 = {e}^{- \frac{1}{2} {\vert - u - β \vert}^{2}} {e}^{\frac{1}{2} ( - {u}^{*} β + u {β}^{*} )} {e}^{- \frac{1}{2} {\vert β - u \vert}^{2}} {e}^{\frac{1}{2} ( {β}^{*} u - β {u}^{*} )} \\ = {e}^{- {\vert u \vert}^{2}} {e}^{- {\vert β \vert}^{2}} {e}^{u {β}^{*} - {u}^{*} β}\end{matrix} \qquad ( 73 )$$

Therefore, substituting Eq. (73) into Eq. (72), the following is obtained

$$\begin{matrix}P ( α ) = \frac{{e}^{{\vert α \vert}^{2}}}{{π}^{2}} \int {e}^{- {α}^{*} u + {u}^{*} α} {e}^{- {\vert u \vert}^{2}} {e}^{- {\vert β \vert}^{2}} {e}^{u {β}^{*} - {u}^{*} β} {e}^{{\vert u \vert}^{2}} {d}^{2} u \\ = \frac{{e}^{{\vert α \vert}^{2}} {e}^{- {\vert β \vert}^{2}}}{{π}^{2}} \int {e}^{- {α}^{*} u + {u}^{*} α} {e}^{u {β}^{*} - {u}^{*} β} {d}^{2} u \\ = \frac{{e}^{{\vert α \vert}^{2}} {e}^{- {\vert β \vert}^{2}}}{{π}^{2}} \int {e}^{- {u ( α - β )}^{*} + {u}^{*} ( α - β )} {d}^{2} u\end{matrix} \qquad ( 74 )$$

and considering that u=x′+iy′, the following is obtained

$$\begin{matrix}P ( α ) = \frac{{e}^{{\vert α \vert}^{2}} {e}^{- {\vert β \vert}^{2}}}{{π}^{2}} \int {e}^{i 2 {x}^{′} Im ( α - β ) - i 2 {y}^{′} Re ( α - β )} {dx}^{′} {dy}^{′} \\ = \frac{{e}^{{\vert α \vert}^{2}} {e}^{- {\vert β \vert}^{2}}}{{( 2 π )}^{2}} \int {e}^{{ix}^{″} Im ( α - β ) - {iy}^{″} Re ( α - β )} {dx}^{″} {dy}^{″} \\ = {e}^{{\vert α \vert}^{2}} {e}^{- {\vert β \vert}^{2}} δ ( Im ( α - β ) ) δ ( Re ( α - β ) ) \\ = {δ}^{2} ( α - β )\end{matrix} \qquad ( 75 )$$

Hence, for a pure coherent state, P(α) coincides with the classical density of states. In particular, this shows that coherent states are classical-like quantum states.

### Pure Number States.

The P-representation of a pure number state, \hat{ρ}=|n [symbol] [symbol] n|, is obtained, as follows

$$\begin{matrix}〈 - u \vert \hat{ρ} \vert u 〉 = 〈 - u \vert n 〉 〈 n \vert u 〉 \\ = {e}^{- {\vert u \vert}^{2}} \frac{{u}^{n}}{n !} {( - {u}^{*} )}^{n}\end{matrix} \qquad ( 76 )$$

where [symbol] n|u [symbol] has been substituted in the second row, according to Eq. (42), as follows

$$〈 n \vert u 〉 = {e}^{- \frac{1}{2} {\vert u \vert}^{2}} \frac{{u}^{n}}{\sqrt{n !}} \qquad ( 77 )$$

Therefore, substituting Eq. (76) into Eq. (72), the following is obtained

$$\begin{matrix}P ( α ) = \frac{{e}^{{\vert α \vert}^{2}}}{{π}^{2}} \int {e}^{- {α}^{*} u + {u}^{*} α} 〈 - u \vert \hat{ρ} \vert u 〉 {e}^{{\vert u \vert}^{2}} {d}^{2} u \\ = \frac{{e}^{{\vert α \vert}^{2}}}{{π}^{2}} \int {e}^{- {α}^{*} u + {u}^{*} α} {e}^{- {\vert u \vert}^{2}} \frac{{( - u {u}^{*} )}^{n}}{n !} {e}^{{\vert u \vert}^{2}} {d}^{2} u \\ = \frac{{e}^{{\vert α \vert}^{2}}}{n ! {π}^{2}} \int {{e}^{- {α}^{*} u + {u}^{*} α} ( - u {u}^{*} )}^{n} {d}^{2} u \\ = \frac{{e}^{{\vert α \vert}^{2}}}{n ! {π}^{2}} \frac{{\partial }^{2 n}}{{\partial }^{n} α {\partial }^{n} {a}^{*}} \int {e}^{- {α}^{*} u + {u}^{*} α} {d}^{2} u\end{matrix} \qquad ( 78 )$$

which can also be written as

$$P ( α ) = \frac{{e}^{{\vert α \vert}^{2}}}{n !} \frac{{\partial }^{2 n}}{{\partial }^{n} α {\partial }^{n} {α}^{*}} {δ}^{2} α \qquad ( 79 )$$

Analogously, for an M-mode Gaussian state, the following is obtained

$${P}_{{⊗}_{j = 1}^{M} \vert {n}_{j} 〉 〈 {n}_{j} \vert} ( α ) = {\prod }_{j = 1}^{M} \frac{{e}^{{\vert {α}_{j} \vert}^{2}}}{{n}_{j} !} \frac{{\partial }^{2 {n}_{j}}}{{\partial }^{{n}_{j}} {α}_{j} {\partial }^{{n}_{j}} {α}_{j}^{*}} {δ}^{2} {α}_{j} \qquad ( 80 )$$

It is noted that this is the so-called tempered distribution function, which operates only as the argument of an integral, as follows

$${\int F ( α ) \frac{{\partial }^{2 n}}{{\partial }^{n} α {\partial }^{n} {α}^{*}} {δ}^{2} α {d}^{2} α = \frac{{\partial }^{2 n} F ( α )}{\partial {α}^{n} {\partial }^{n} {α}^{*}} \vert}_{α = 0 , {α}^{*} = 0} \qquad ( 81 )$$

### Expectation Values for Operators.

In general, the P-representation of an operator Ô(â^{†}, â), is analogous to the representation of the density operator introduced by Eq. (65). It involves the P-function P_{Ô}(α), which is defined, as follows

$$\hat{O} = \int {P}_{\hat{O}} ( α ) \vert α 〉 〈 α \vert {d}^{2} α \qquad ( 82 )$$

with expectation value given by

$$\begin{matrix}〈 \hat{O} 〉 = Tr [ \hat{O} \hat{ρ} ] \\ = {\sum }_{n} \int {P}_{\hat{O}} ( α ) 〈 n \vert α 〉 〈 α \vert \hat{ρ} \vert n 〉 {d}^{2} α \\ = \int {P}_{\hat{O}} ( α ) {\sum }_{n} 〈 α \vert \hat{ρ} \vert n 〉 〈 n \vert α 〉 {d}^{2} α \\ = \int {P}_{\hat{O}} ( α ) 〈 α \vert \hat{ρ} \vert α 〉 {d}^{2} α \\ = π \int {P}_{\hat{O}} ( α ) Q ( α ) {d}^{2} α\end{matrix} \qquad ( 83 )$$

where Q(α)=π⁻¹ [symbol] α|\hat{ρ}|α [symbol] is called the Husimi function. In particular, for a pure state \hat{ρ}=|ψ [symbol] [symbol] ψ|, the Husimi function is Q(α)=π⁻¹| [symbol] ψ|α [symbol] |². The derivation of the Husimi function for a multimode Gaussian will be the subject of the following subsection. For the particular case Ô=|n [symbol] [symbol] n|, the following is obtained:

$$Tr [ \hat{O} \hat{ρ} ] = Tr [ \hat{ρ} \vert n 〉 〈 n \vert ] = π \int {P}_{\vert n 〉 〈 n \vert} ( α ) Q ( α ) {d}^{2} α \qquad ( 84 )$$

where [symbol] (α) is defined according to Eq. (79) as

$${P}_{\vert n 〉 〈 n \vert} ( α ) = \frac{{e}^{{\vert α \vert}^{2}}}{n !} \frac{{\partial }^{2 n}}{{\partial }^{n} α {\partial }^{n} {α}^{*}} {δ}^{2} α \qquad ( 85 )$$

which yields

$$Tr [ \hat{ρ} \vert n 〉 〈 n \vert ] = π \int Q ( α ) \frac{{e}^{{\vert α \vert}^{2}}}{n !} \frac{{\partial }^{2 n}}{{\partial }^{n} α {\partial }^{n} {α}^{*}} {δ}^{2} α {d}^{2} α \qquad ( 86 )$$

### Husimi Function of an M-Mode Gaussian State

The Husimi function of an M-mode Gaussian state is defined, as follows

$$Q ( α ) = {π}^{- M} 〈 α \vert \hat{ρ} \vert α 〉 \qquad ( 87 )$$

where α=(α₁, . . . , α_{M}, α₁*, . . . , α_{M}*)^{T}. The goal of this subsection is to introduce the Wigner transform for the evaluation of Eq. (87) for M=1. As a result of this, the following expression will be obtained

$$Q ( α ) = \frac{{π}^{- 1}}{\sqrt{\vert det ( {σ}_{Q} ) \vert}} {e}^{- {α}^{†} {σ}_{Q}^{- 1} α} \qquad ( 88 )$$

$$where$$

$${σ}_{Q} = σ + {I}_{2} / 2.$$

### Wigner Transform.

For the derivation of Eq. (88), the Husimi function for a single-mode pure Gaussian state is first given. The elements of the density operator of a single-mode pure Gaussian state are given by

$$\begin{matrix}〈 x \vert \hat{ρ} \vert {x}^{′} 〉 = 〈 x \vert ψ 〉 〈 ψ \vert {x}^{′} 〉 \\ = {( \frac{γ}{π} )}^{1 / 2} {e}^{- \frac{γ}{2} ( {( x - {d}_{x} )}^{2} + {( {x}^{′} - {d}_{x} )}^{2} ) + \frac{i}{ℏ} {d}_{p} ( x - {x}^{′} )}\end{matrix} \qquad ( 89 )$$

where dₓ= [symbol] \hat{x} [symbol] , dₚ= [symbol] \hat{p} [symbol] , and γ=( [symbol] (\hat{x}−dₓ)² [symbol] )⁻¹/2. These elements can be Wigner transformed, as follows

$$\begin{matrix}{ρ}_{W} ( x , p ) = {( 2 π ℏ )}^{- 1} \int {e}^{\frac{i}{ℏ} py} 〈 x - \frac{y}{2} \vert \hat{ρ} \vert x + \frac{y}{2} 〉 dy \\ = {( 2 πℏ )}^{- 1} \int {( \frac{γ}{π} )}^{1 / 2} {e}^{- \frac{γ}{2} ( {( x - \frac{y}{2} - {d}_{x} )}^{2} + {( x + \frac{y}{2} - {d}_{x} )}^{2} ) + \frac{i}{ℏ} ( p - {d}_{p} ) y} dy \\ = {( 2 πℏ )}^{- 1} {e}^{- γ ( {x}^{2} - 2 {xd}_{x} + {d}_{x}^{2} )} \int {( \frac{γ}{π} )}^{1 / 2} {e}^{- \frac{γ}{4} {y}^{2} + \frac{i}{ℏ} ( p - {d}_{p} ) y} dy \\ = {( π ℏ )}^{- 1} {e}^{- ( {γ ( x - {d}_{x} )}^{2} + {( p - {d}_{p} )}^{2} / ( {ℏ}^{2} γ ) )}\end{matrix} . \qquad ( 90 )$$

For a given positive constant γ_{α}>0, let z=(\sqrt{γ_{α}}(x−dₓ),(p−dₚ)/(p−dₚ)/(ℏ\sqrt{γ_{α}}))^{T}, and

$$σ = [ \begin{matrix}{γ}_{α} / γ & 0 \\ 0 & γ / {γ}_{α}\end{matrix} ] \qquad ( 91 )$$

Then, the following can be obtained

$${ρ}_{W} ( x , p ) = \frac{{e}^{- {z}^{T} {σ}^{- 1} z}}{π ℏ} = \frac{{e}^{- \frac{1}{2} {z}^{T} {\tilde{σ}}^{- {1}_{z}}}}{2 π ℏ \sqrt{\vert det ( \tilde{σ} \vert )}} \qquad ( 92 )$$

$$where$$

$${\tilde{σ}}^{- 1} = 2 {σ}^{- 1} .$$

### Expectation Values.

Expectation values can be calculated in terms of the Wigner transform, as follows

$$\begin{matrix}〈 \hat{O} 〉 = Tr [ \hat{ρ} \hat{O} ] \\ \int 〈 \tilde{x} \vert \hat{ρ} \vert {\tilde{x}}^{′} 〉 〈 {\hat{x}}^{′} \vert \hat{O} \vert \tilde{x} 〉 d \tilde{x} d {\tilde{x}}^{′} \\ \int 〈 x - \frac{y}{2} \vert \hat{ρ} \vert x + \frac{y}{2} 〉 〈 x + \frac{y}{2} \vert \hat{O} \vert x - \frac{y}{2} 〉 dx dy\end{matrix} \qquad ( 93 )$$

where

$$\tilde{x} = x - \frac{y}{2}$$

$$and$$

$${\tilde{x}}^{′} = x + \frac{y}{2} .$$

### Therefore,

$$( 94 )$$

$$\begin{matrix}〈 \hat{O} 〉 = \int 〈 x - \frac{y}{2} \vert \hat{ρ} \vert x + \frac{y}{2} 〉 \int 〈 x + \frac{{y}^{′}}{2} \vert \hat{O} \vert x - \frac{{y}^{′}}{2} 〉 δ ( y - {y}^{′} ) {dy}^{′} dx dy \\ = \frac{1}{2 π ℏ} \int \int 〈 x - \frac{y}{2} \vert \hat{ρ} \vert x + \frac{y}{2} 〉 {e}^{\frac{1}{ℏ} py} \int 〈 x + \frac{{y}^{′}}{2} \vert \hat{O} \vert x - \frac{{y}^{′}}{2} 〉 {e}^{- \frac{i}{ℏ} {py}^{′}} {dy}^{′} dy dx dp \\ = \int {ρ}_{W} ( x , p ) \int 〈 x + \frac{{y}^{′}}{2} \vert \hat{O} \vert x - \frac{{y}^{′}}{2} 〉 {e}^{- \frac{i}{ℏ} {py}^{′}} {dy}^{′} dx dp \\ = \int {ρ}_{W} ( x , p ) \int 〈 x + \frac{{y}^{′}}{2} \vert \hat{O} \vert x - \frac{{y}^{′}}{2} 〉 {e}^{\frac{i}{ℏ} {py}^{′}} {dy}^{′} dx dp \\ = \int {ρ}_{W} ( x , p ) {O}_{W} ( x , p ) dx dp\end{matrix}$$

where

$${O}_{W} ( x , p ) = \int 〈 x - \frac{{y}^{′}}{2} \vert \hat{O} \vert x - \frac{{y}^{′}}{2} 〉 {e}^{\frac{i}{ℏ} {py}^{′}} {dy}^{′} .$$

In particular, the expectation value [symbol] α|\hat{\hat{ρ}}|α [symbol] is given by

$$〈 α \vert \hat{ρ} \vert α 〉 = \int {ρ}_{W} ( x , p ) {W}_{α} ( x , p ) dx dp \qquad ( 95 )$$

with |α [symbol] defined according to Eq. (63), and

$$( 96 )$$

$${W}_{α} ( x , p ) = \int 〈 x - \frac{{y}^{′}}{2} \vert α 〉 〈 α \vert x + \frac{{y}^{′}}{2} 〉 {e}^{\frac{i}{ℏ} {py}^{′}} {dy}^{′} = 2 {e}^{- {{γ}_{α} ( x - {q}_{α} )}^{2}} {e}^{- {( p - {p}_{α} )}^{2} / ( {γ}_{α} {ℏ}^{2} )}$$

with γ_{α}=mω/ℏ,q_{α}= [symbol] α|\hat{x}|α [symbol] , and p_{α}= [symbol] α|\hat{p}|α [symbol] , corresponding to the value α=\sqrt{γ_{α}/2}q_{α}+i(2γ_{α})^{-1/2}ℏ⁻¹p_{α}, according to Eq. (59). Analogously, β=\sqrt{γ_{α}/2}x+i(2γ_{α})^{-1/2}ℏ⁻¹p is introduced, from which it is concluded that

$$2 {\vert β - α \vert}^{2} = {{γ}_{α} ( x - {q}_{α} )}^{2} + {( p - {p}_{α} )}^{2} / ( {γ}_{a} {ℏ}^{2} ) \qquad ( 97 )$$

which shows that

$${W}_{α} ( x , p ) = 2 {e}^{- 2 {\vert β - α \vert}^{2}} \qquad ( 98 )$$

Substituting Eqs. (92) and (98) into Eq. (95), it is found:

$$〈 α \vert \hat{ρ} \vert α 〉 = \int \frac{{e}^{- \frac{1}{2} {z}^{T} {\tilde{σ}}^{- 1} z}}{π ℏ \sqrt{\vert det ( \tilde{σ} ) \vert}} {e}^{- 2 {\vert β - α \vert}^{2}} dx dp = \frac{1}{π \sqrt{\vert det ( \tilde{σ} ) \vert}} \int {e}^{- \frac{1}{2} {β}^{†} {\tilde{σ}}^{- 1} β} {e}^{- 2 {\vert β - α \vert}^{2}} {d}^{2} β \qquad ( 99 )$$

For simplicity, consider the case where dₓ=dₚ=0. Using that |β−α|²=(β−α)^{†}(β−α)=β^{†}β−β^{†}α−α^{†}β+α^{†}α=β^{†}β−2α^{†}β+α^{†}α, the following is obtained

$$\begin{matrix}〈 α \vert \hat{ρ} \vert α 〉 = \frac{{e}^{- 2 {α}^{†} α}}{π \sqrt{\vert det ( \tilde{σ} ) \vert}} \int {e}^{- \frac{1}{2} {β}^{†} ( {\tilde{σ}}^{- 1} + 4 ) β} {e}^{4 {α}^{†} β} {d}^{2} β \\ = \frac{2 {e}^{- 2 {α}^{†} α}}{\sqrt{\vert det ( {\tilde{σ}}^{- 1} + 4 ) \vert \vert det ( \tilde{σ} ) \vert}} {e}^{\frac{1}{2} 4 {{α}^{†} ( {\tilde{σ}}^{- 1} + 4 )}^{- 1} 4 α} \\ = \frac{2 {e}^{- {α}^{†} ( 2 - 8 {( {\tilde{σ}}^{- 1} + 4 )}^{- 1} ) α}}{\sqrt{\vert det ( {\tilde{σ}}^{- 1} + 4 ) \vert \vert det ( \tilde{σ} ) \vert}}\end{matrix} \qquad ( 100 )$$

Moreover, a short calculation shows that 2−8 (\tilde{σ}⁻¹+4)⁻¹=(σ+I₂/2)⁻¹, as well as \sqrt{|det(\tilde{σ}⁻¹+4)∥det(\tilde{σ})}=2\sqrt{|det(I₂/2+σ)|}. Therefore, it is finally concluded that

$$Q ( α ) = {π}^{- 1} 〈 α \vert \hat{ρ} \vert α 〉 = {π}^{- 1} \frac{{e}^{- {{α}^{†} ( σ + {I}_{2} / 2 )}^{- 1} α}}{\sqrt{\vert det ( σ + {I}_{2} / 2 ) \vert}} \qquad ( 101 )$$

which, when generalized to an M-mode Gaussian, gives Eq. (88).

### Output Probabilities of an M-Mode Gaussian

In this section, the output probability distribution is obtained

$$Pr ( \overline{n} ) = Tr [ \hat{ρ} {⊗}_{j = 1}^{M} \vert {n}_{j} 〉 〈 {n}_{j} \vert ] \qquad ( 102 )$$

for an M-mode input Gaussian, corresponding to occupation numbers of output modes ñ as described by the tensor product of number state operators \overline{\hat{n}}=⊗ⱼ₌₁^{M}\hat{n}ⱼ, where \hat{n}ⱼ=|nⱼ [symbol] [symbol] nⱼ| measures the probability of observing nⱼ photons in output mode j.

Substituting Eq. (88) and Eq. (85) into Eq. (84), the following is obtained

$$Pr ( \overline{n} ) = \int \frac{1}{\sqrt{\vert det ( {σ}_{Q} ) \vert}} {e}^{- \frac{1}{2} {α}^{†} {σ}_{Q}^{- 1} α + {α}^{†} α} {\prod }_{j = 1}^{M} \frac{1}{{n}_{j} !} \frac{{\partial }^{2 {n}_{j}}}{{\partial }^{{n}_{j}} {α}_{j} {\partial }^{{n}_{j}} {α}_{j}^{*}} {δ}^{2} {α}_{j} \qquad ( 103 )$$

and integration by parts yields

$${Pr ( \overline{n} ) = \frac{1}{\sqrt{\vert det ( {σ}_{Q} ) \vert}} {\prod }_{j = 1}^{M} \frac{1}{{n}_{j} !} \frac{{\partial }^{2 {n}_{j}}}{{\partial }^{{n}_{j}} {α}_{j} {\partial }^{{n}_{j}} {α}_{j}^{*}} {e}^{\frac{1}{2} {α}^{†} ( {I}_{2 M} - {σ}_{Q}^{- 1} ) α} \vert}_{{α}_{j} = 0} \qquad ( 104 )$$

It is noted that

$${α}^{†} ( {I}_{2 M} - {σ}_{Q}^{- 1} ) α = {α}^{T} [ \begin{matrix}0 & {I}_{M} \\ {I}_{M} & 0\end{matrix} ] ( {I}_{2 M} - {σ}_{Q}^{- 1} ) α \qquad ( 105 )$$

since is defined as α=(α₁, . . . , α_{M}, α₁*, . . . , α_{M}*)^{T}. Hence,

$${Pr ( \overline{n} ) = \frac{1}{\sqrt{\vert det ( {σ}_{Q} ) \vert}} {\prod }_{j = 1}^{M} \frac{1}{{n}_{j} !} \frac{{\partial }^{2 {n}_{j}}}{{\partial }^{{n}_{j}} {α}_{j} {\partial }^{{n}_{j}} {α}_{j}^{*}} {e}^{\frac{1}{2} {α}^{T} K α} \vert}_{{α}_{j} = 0} , \qquad ( 106 )$$

$$where$$

$$K = [ \begin{matrix}0 & {I}_{M} \\ {I}_{M} & 0\end{matrix} ] ( {I}_{2 M} - {σ}_{Q}^{- 1} ) \qquad ( 107 )$$

was used as previously defined. In particular, for the specific case of measuring nⱼ∈{0,1} photons at each output mode, with N=Σⱼ₌₁^{M}nⱼ, the following is obtained

$$( 108 )$$

$${{Pr ( \overline{n} ) = \frac{1}{\sqrt{\vert det ( {σ}_{Q} ) \vert}} \frac{{\partial }^{2 N}}{{\prod }_{j = 1}^{M} {\partial }^{{n}_{j}} {α}_{j} {\partial }^{{n}_{j}} {α}_{j}^{*}} {e}^{\frac{1}{{α}^{T}} K α} \vert}_{{α}_{j} = 0} = \frac{1}{\sqrt{\vert det ( {σ}_{Q} ) \vert}} \frac{{\partial }^{2 N}}{{\prod }_{j = 1}^{M} {\partial }^{{n}_{j}} {α}_{j} {\partial }^{{n}_{j}} {α}_{j}^{*}} {e}^{\frac{1}{{α}^{T}} K α} \vert}_{{α}_{j} = 0}$$

where in the second row the indices l=1, . . . , N correspond to the output modes with nⱼ=1. To evaluate Eq. (108), Faà di Bruno's formula in the section titled “Faà di Bruno's formula,” showing that it can be evaluated, as follows

$$Pr ( \overline{n} ) = \frac{1}{\sqrt{\vert det ( {σ}_{Q} ) \vert}} {\sum }_{j = 1}^{( 2 N - 1 ) ! !} {\prod }_{k = 1}^{N} {K}_{{μ}_{j} ( 2 k - 1 ) , {μ}_{j} ( 2 k )} = \frac{1}{\sqrt{\vert det ( {σ}_{Q} ) \vert}} Haf ( {K}_{s} ) \qquad ( 109 )$$

where μⱼ∈S_{2N} (symmetric group of 2N elements) define the indices of measured photons (bright modes) corresponding to perfect matching j (of which there exist (2N)!/(2^{N}N!)=(2N−1)!!), and K_{S} is the submatrix of K corresponding to the indices of measured photons.

It is observed that Pr (n̅) is directly proportional to the number of perfect matchings associated with the observed modes with indices n when A is the adjacency matrix of the complete graph of modes, K=A^{⊕2}=c(A⊕A), and K_{S} the submatrix of K corresponding to the observed modes. Therefore, the number of perfect matchings can be obtained by sampling from a Gaussian distribution with covariance σ_{Q}=σ+I_{2M}/2.

### Faà di Bruno's Formula

Equation (106) can be evaluated using Faà di Bruno's formula

$$\frac{{\partial }^{n}}{\partial {x}_{1} … \partial {x}_{n}} f ( y ) = {\sum }_{π ∈ {P}_{n}} {f}^{( \vert π \vert )} ( y ) {\prod }_{B ∈ π} \frac{{\partial }^{\vert B \vert} y}{{\prod }_{l ∈ B} \partial {x}_{l}} , \qquad ( 110 )$$

$$y = y ( {x}_{1} , {x}_{2} , … , {x}_{n} )$$

where Pₙ is the set of partitions of n indices {1, 2, . . . , n}, while |π| is the number of blocks of partition π, and |B| is the number of elements in block B.

A simple example with n=3 illustrates Eq. (110) as applied to computing

$$\frac{{\partial }^{3}}{\partial {x}_{1} \partial {x}_{2} \partial {x}_{3}} f ( y ) ,$$

with y=y(x₁, x₂, x₃). The set P₃ of possible partitions of indices {1,2,3} includes one partition π₁ with one block π₁={{1,2,3}}(|π₁|=1, one block of size three); three partitions with two blocks, including π₂={{1,2}, {3}}, π₃={{1,3}, {2}}, π₄={{2,3}, {1}} (|π_{2,3,4}|=2); and finally one partition with three blocks π₅={{1}, {2}, {3}}(|π₅|=3). Hence, |P₃|=5 and the following is obtained

$$\frac{{\partial }^{3}}{\partial {x}_{1} \partial {x}_{2} \partial {x}_{3}} f ( y ) = {f}^{′} ( y ) \frac{{\partial }^{3} y}{\partial {x}_{1} \partial {x}_{2} \partial {x}_{3}} + … {f}^{″} ( y ) ( \frac{{\partial }^{2} y}{\partial {x}_{1} \partial {x}_{2}} \frac{\partial y}{\partial {x}_{3}} + \frac{{\partial }^{2} y}{\partial {x}_{1} \partial {x}_{3}} \frac{\partial y}{\partial {x}_{2}} + \frac{{\partial }^{2} y}{\partial {x}_{2} \partial {x}_{3}} \frac{\partial y}{\partial {x}_{1}} ) + … {f}^{″′} ( y ) \frac{\partial y}{\partial {x}_{1}} \frac{\partial y}{\partial {x}_{2}} \frac{\partial y}{\partial {x}_{3}} \qquad ( 111 )$$

Analogously, to evaluate Eq. (108), n=2N,

$$x = α ,$$

$$y = \frac{1}{2} {α}^{T} K α ,$$

and f(y)=e^{y} are used. Note that in this case f^{(|π|)}(y)=f(y) for all partitions. It is important to note that the function

$$\frac{1}{2} {α}^{T} K α$$

is quadratic in α, so an derivatives of third order or higher vanish. Furthermore, the argument of Eq. (108) is evaluated at αₗ=0, so all first order derivatives also vanish. This leaves only the partitions for which |B|=2 for all blocks, which implies |π|=N (i.e., perfect matchings, the partitions of 2N indices into N blocks of pairs can be interpreted as permutations of the 2N photon indices). Therefore, the following is obtained

$$\frac{{\partial }^{n}}{\partial {x}_{1} … \partial {x}_{n}} f ( y ) = \frac{{\partial }^{2 N} {e}^{\frac{1}{2} {α}^{T} K α}}{{\prod }_{l = 1}^{N} \partial {α}_{l} \partial {α}_{l}^{*}} = {e}^{\frac{1}{2} {α}^{T} K α} {\sum }_{π ∈ PM} {\prod }_{B ∈ π} \frac{{\partial }^{2} y}{{\prod }_{l ∈ B} \partial {x}_{l}} \qquad ( 112 )$$

where PM⊂P_{2N} denotes the subset of perfect matchings. Using that the second-order derivatives of y can be expressed in terms of the matrix elements of K, the following is obtained

$$\begin{matrix}{Pr ( \overline{n} ) = \frac{1}{\sqrt{\vert det ( {σ}_{Q} ) \vert}} \frac{{\partial }^{2 N}}{{\prod }_{l = 1}^{N} \partial {α}_{l} \partial {α}_{l}^{*}} {e}^{\frac{1}{2} {α}^{T} K α} \vert}_{{α}_{l} = 0} \\ = \frac{1}{\sqrt{\vert det ( {σ}_{Q} ) \vert}} {\sum }_{j = 1}^{( 2 N - 1 ) ! !} {\prod }_{k = 1}^{N} {K}_{{μ}_{j} ( 2 k - 1 ) , {μ}_{j} ( 2 k )} \\ = \frac{1}{\sqrt{\vert det ( {σ}_{Q} ) \vert}} Haf ( {K}_{s} )\end{matrix} \qquad ( 113 )$$

A simple example, with N=2, shows how the partitions that contribute to Eq. (112) can be interpreted as permutations of the photon indices. Assume that M=4 and N=2 photons are measured in the last two output modes 3 and 4, providing α={α₃, α₄, α₃*, α₄*}. The indices are labeled as follows: {α₃→1, α₄→2, α₃*→3, α₄*→4}. The perfect matchings are given by

$${π}_{{PM}_{1}} = \{ \{ 1 , 2 \} , \{ 3 , 4 \} \}$$

$${π}_{{PM}_{2}} = \{ \{ 1 , 3 \} , \{ 2 , 4 \} \}$$

$${π}_{{PM}_{3}} = \{ \{ 1 , 4 \} , \{ 2 , 3 \} \}$$

It is therefore concluded that

$$\begin{matrix}{Pr ( \overline{n} ) = \frac{1}{\sqrt{\vert det ( {σ}_{Q} ) \vert}} \frac{{\partial }^{2 N}}{{\prod }_{l = 1}^{N} \partial {α}_{l} \partial {α}_{l}^{*}} {e}^{\frac{1}{2} {α}^{T} K α} \vert}_{{α}_{l} = 0} \\ = \frac{1}{\sqrt{\vert det ( {σ}_{Q} ) \vert}} {\sum }_{j = 1}^{( 2 N - 1 ) ! !} {\prod }_{k = 1}^{N} {K}_{{μ}_{j} ( 2 k - 1 ) , {μ}_{j} ( 2 k )} \\ = \frac{1}{\sqrt{\vert det ( {σ}_{Q} ) \vert}} Haf ( {K}_{s} )\end{matrix} \qquad ( 113 )$$

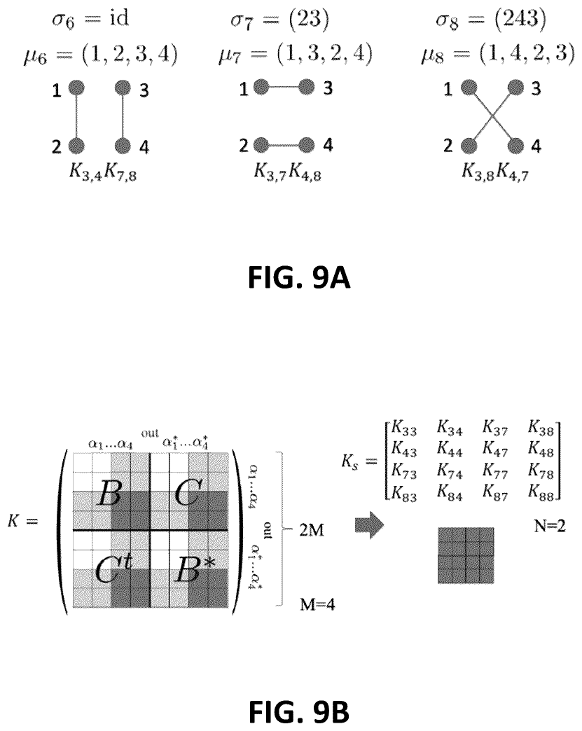

It is noted that the index combinations of the partial derivatives in the first row can be mapped into permutations corresponding to perfect matchings. These permutations (FIGS. 9A-9B) can be written in vector format as μ₁=(1,2,3,4), μ₂=(1,3,2,4) and μ₃=(1,4,2,3), also denoted as σ₁=id, σ₂=(23), and σ₃=(243). Note, that the blocks are ordered from left to right corresponding to their block indices from lowest to highest and the numbers within a block are also in increasing order, so μⱼ(2k−1)<μⱼ(2k+1) and μⱼ(2k−1)<μⱼ(2k).

### Covariance Matrix of Squeezed and Rotated State

The goal of sections “Covariance matrix of squeezed and rotated state” and “Kernel matrix for the N-mode rotated state” is to prove Eq. (13), which establishes a relationship between the covariance matrix of the M-mode squeezed and rotated state, σ, and the graph adjacency matrix A. Here, the expression of σ expressed according to the squeezing parameters and the M-mode rotation matrix U that defines the linear interferometer are given. Then in “Kernel matrix for the N-mode rotated state” it is shown how A^{⊕2}=cA⊕cA is identified as the so-called kernel matrix K, which one-to-one correspond to the covariance matrix σ.

Recall the definition of the squeezing operation

$$\hat{S} ( r ) = {e}^{( - {r ( \hat{a} )}^{2} + {r ( {\hat{a}}^{†} )}^{2} ) / 2} \qquad ( 115 )$$

where the real-valued r is referred to as the squeezing parameter. It is aimed to show that the action of the squeezing operator on the creation and annihilation operators are equivalent to the following linear Bogoliubov transformation

$$[ \begin{matrix}\hat{a} \\ {\hat{a}}^{†}\end{matrix} ] ↦ [ \begin{matrix}cosh ( r ) & sinh ( r ) \\ sinh ( r ) & cosh ( r )\end{matrix} ] [ \begin{matrix}\hat{a} \\ {\hat{a}}^{†}\end{matrix} ] = [ \begin{matrix}{\hat{a}}^{′} \\ {( {\hat{a}}^{′} )}^{†}\end{matrix} ] . \qquad ( 116 )$$

To proof this relationship, the following is derived

$${\hat{S} ( r )}^{†} \hat{a} \hat{S} ( r ) = cosh ( r ) \hat{a} + sinh ( r ) {\hat{a}}^{†} \qquad ( 117 )$$

The following is set

$$Â = ( {r ( \hat{a} )}^{2} - {( {\hat{a}}^{†} )}^{2} ) / 2 \qquad ( 118 )$$

so that Ŝ(r)^{†}=e^{Â}. Then, using the Baker-Campbell-Hausdorff formula, the following is obtained

$${\hat{S} ( r )}^{†} \hat{a} \hat{S} ( r ) = {e}^{Â} \hat{a} {e}^{- Â}$$

where each term contains k commutators. Noting that

$$[ Â , \hat{a} ] = \frac{1}{2} [ {r ( \hat{a} )}^{2} - {r ( {\hat{a}}^{†} )}^{2} , \hat{a} ] = r {\hat{a}}^{†} \qquad ( 120 )$$

$$[ Â , {\hat{a}}^{†} ] = r \hat{a}$$

$$then$$

$$[ Â , … [ Â , \hat{a} ] … ] ] = \{ \begin{matrix}{r}^{k} \hat{a} & if k is even \\ {r}^{k} {\hat{a}}^{†} & if k is odd\end{matrix} \qquad ( 121 )$$

Then the following is obtained

$${\hat{S} ( r )}^{†} \hat{a} \hat{S} ( r ) = \hat{a} {\sum }_{k = 0}^{∞} \frac{{r}^{2 k}}{( 2 k ) !} + {\hat{a}}^{†} {\sum }_{k = 0}^{∞} \frac{{r}^{2 k + 1}}{( 2 k + 1 ) !} \qquad ( 122 )$$

It is now shown that the covariance matrix of single mode squeezed state is

$${σ}^{′} ( r ) = \frac{1}{2} [ \begin{matrix}{cosh}^{2} ( r ) + {sinh}^{2} ( r ) & 2 cosh ( r ) sinh ( r ) \\ 2 cosh ( r ) sinh ( r ) & {cosh}^{2} ( r ) + {sinh}^{2} ( r )\end{matrix} ] \qquad ( 123 )$$

For the first element of the squeezed state covariance matrix σ₁₁′, the definition of the covariance matrix in Eq. (3) is substituted in

$$( 124 )$$

$$\begin{matrix}{σ}_{11}^{′} = \frac{1}{2} 〈 \{ {\hat{a}}^{′} , {( {\hat{a}}^{′} )}^{†} \} 〉 \\ = \frac{1}{2} 〈 {{\hat{a}}^{′} ( {\hat{a}}^{′} )}^{†} + {( {\hat{a}}^{′} )}^{†} {\hat{a}}^{′} 〉 \\ = \frac{1}{2} 〈 ( cosh {( r )}^{2} + sinh {( r )}^{2} ) ( \hat{a} {\hat{a}}^{†} + {\hat{a}}^{†} \hat{a} ) + 2 sinh ( r ) cosh ( r ) ( \hat{a} \hat{a} + {\hat{a}}^{†} {\hat{a}}^{†} ) 〉 \\ = \frac{1}{2} ( cosh {( r )}^{2} + sinh {( r )}^{2} ) 〈 \hat{a} {\hat{a}}^{†} + {\hat{a}}^{†} \hat{a} 〉 + sinh ( r ) cosh ( r ) 〈 ( \hat{a} \hat{a} + {\hat{a}}^{†} {\hat{a}}^{†} ) 〉 \\ = \frac{1}{2} ( cosh {( r )}^{2} + sinh {( r )}^{2} )\end{matrix}$$

where the last equality uses [symbol] ââ^{†}+â^{†}â [symbol] =1 and [symbol] ââ [symbol] = [symbol] â^{†}â^{†} [symbol] =0 for the vacuum state. Note that all averages are taken for the vacuum state density matrix since the creation and annihilation operators are in the Heisenberg representation. Similarly,

$$\begin{matrix}{σ}_{12}^{′} = \frac{1}{2} 〈 2 {\hat{a}}^{′} {\hat{a}}^{′} 〉 \\ = 〈 ( cosh ( r ) \hat{a} + sinh ( r ) {\hat{a}}^{†} ) ( cosh ( r ) \hat{a} + sinh ( r ) {\hat{a}}^{†} ) 〉 \\ = 〈 cosh {( r )}^{2} ( \hat{a} \hat{a} ) + sinh {( r )}^{2} ( {\hat{a}}^{†} {\hat{a}}^{†} ) + 2 sinh ( r ) cosh ( r ) \{ {\hat{a}}^{†} , \hat{a} \} 〉 \\ = sinh ( r ) cosh ( r )\end{matrix} \qquad ( 125 )$$

The derivation for the other two elements of σ′ are analogous. Gathering all elements Eq. (123) is obtained.

Next, it is shown that applying an M-mode rotation, specified by an M×M unitary rotation matrix U, on the single-mode squeezed states specified by Eq. (123), results in a state with the following covariance matrix

$$σ = [ \begin{matrix}U & 0 \\ 0 & {U}^{*}\end{matrix} ] {{σ}_{sque} [ \begin{matrix}{U}^{*} & 0 \\ 0 & U\end{matrix} ]}^{T} \qquad ( 126 )$$

where σ_{sque} is the generalization of Eq. (123) to M modes, as shown in Eq. (24). To prove this, a given 2M×2M covariance matrix \tilde{σ} is written in the following block form

$$\tilde{σ} = [ \begin{matrix}B & G \\ D & C\end{matrix} ] \qquad ( 127 )$$

where all four blocks are M×M matrices. It is now shown that an M-mode rotation specified by the M×M unitary matrix U would rotate the covariance matrix to be

$${\tilde{σ}}^{′} = [ \begin{matrix}U & 0 \\ 0 & {U}^{*}\end{matrix} ] {\tilde{σ} [ \begin{matrix}{U}^{*} & 0 \\ 0 & U\end{matrix} ]}^{T} = [ \begin{matrix}{B}^{′} & {G}^{′} \\ {D}^{′} & {C}^{′}\end{matrix} ] \qquad ( 128 )$$

where B′=UBU^{†}, C′=U*CU^{T}, G′=UGU^{T}, D′=U*DU^{†}. As derived in Eq. (28), the M-mode rotation linearly combines all M original annihilation/creation operators to obtain the rotated annihilation/creation operators, with the combination coefficients specified by elements of

$${\hat{a}}_{k}^{′} = {\sum }_{j = 1}^{M} {U}_{kj} {\hat{a}}_{j} \qquad ( 129 )$$

$${( {\hat{a}}_{k}^{′} )}^{†} = {\sum }_{j = 1}^{M} {U}_{kj}^{*} {\hat{a}}_{j}^{†}$$

According to the definition of the covariance matrix in Eq. (3), an element σᵢⱼ′ of the upper-left block B(1≤i, j≤M) of the rotated covariance matrix σ′, can be expanded as

$$\begin{matrix}{\hat{σ}}_{ij}^{′} = 〈 \{ {{\hat{a}}_{i}^{′} ( {\hat{a}}_{j}^{′} )}^{†} \} 〉 / 2 \\ = 〈 {{\hat{a}}_{i}^{′} ( {\hat{a}}_{j}^{′} )}^{†} + {( {\hat{a}}_{j}^{′} )}^{†} {\hat{a}}_{i}^{′} 〉 / 2 \\ = \frac{1}{2} 〈 ( {\sum }_{l = 1}^{M} {U}_{il} {\hat{a}}_{l} ) ( {\sum }_{m = 1}^{M} {U}_{jm}^{*} {\hat{a}}_{m}^{†} ) + ( {\sum }_{m = 1}^{M} {U}_{jm}^{*} {\hat{a}}_{m}^{†} ) ( {\sum }_{l = 1}^{M} {U}_{il} {\hat{a}}_{l} ) 〉 \\ = \frac{1}{2} {\sum }_{l , m = 1}^{M} {U}_{il} {U}_{jm}^{*} 〈 {\hat{a}}_{l} {\hat{a}}_{m}^{†} + {\hat{a}}_{m}^{†} {\hat{a}}_{l} 〉 \\ = {\sum }_{l , m = 1}^{M} {U}_{il} {\tilde{σ}}_{lm} {U}_{mj}^{†} \\ = {( {UBU}^{†} )}_{ij}\end{matrix} \qquad ( 130 )$$

which proves B′=UBU^{†}. The same procedure is carried out for C, G and D to prove Eq. (128). In particular, with \tilde{σ}=σ_{sque}, the following is obtained

$${σ}_{out} = [ \begin{matrix}U & 0 \\ 0 & {U}^{*}\end{matrix} ] {{σ}_{sque} [ \begin{matrix}{U}^{*} & 0 \\ 0 & U\end{matrix} ]}^{T} as in Eq . ( 30 ) . \qquad ( 131 )$$

### Kernel Matrix for the N-Mode Rotated State

The goal of this section is to prove Eq. (13). First, it is shown that the squeezed state covariance matrix σ′(r), derived as in Eq. (123), corresponds to the kernel matrix K(r) that has the following form

$$K ( r ) = [ \begin{matrix}tanh ( r ) & 0 \\ 0 & tanh ( r )\end{matrix} ] \qquad ( 132 )$$

where the kernel matrix K of a Gaussian state is defined according to its covariance matrix:

$${σ}_{Q} = σ + I / 2 \qquad ( 133 )$$

$$K = X ( I - {σ}_{Q}^{- 1} )$$

$$X = [ \begin{matrix}0 & 1 \\ 1 & 0\end{matrix} ]$$

### According to Eqs. (123) and (133),

$$\begin{matrix}{σ}_{Q}^{′} ( r ) & = {σ}^{′} ( r ) + I / 2 \\ & = \frac{1}{2} [ \begin{matrix}{cosh}^{2} ( r ) + {sinh}^{2} ( r ) + 1 & 2 cosh ( r ) sinh ( r ) \\ 2 cosh ( r ) sinh ( r ) & {cosh}^{2} ( r ) + {sinh}^{2} ( r ) + 1\end{matrix} ] \\ & = [ \begin{matrix}{cosh}^{2} ( r ) & cosh ( r ) sinh ( r ) \\ cosh ( r ) sinh ( r ) & {cosh}^{2} ( r )\end{matrix} ]\end{matrix} \qquad ( 134 )$$

Taking the inverse of the matrix,

$$\begin{matrix}{( {σ}_{Q}^{′} ( r ) )}^{- 1} & = \frac{1}{\begin{matrix}{cosh}^{4} ( r ) - \\ {sinh}^{2} ( r ) {cosh}^{2} ( r )\end{matrix}} [ \begin{matrix}{cosh}^{2} ( r ) & - cosh ( r ) sinh ( r ) \\ - cosh ( r ) sinh ( r ) & {cosh}^{2} ( r )\end{matrix} ] \\ & = \frac{1}{{cosh}^{2} ( r )} [ \begin{matrix}{cosh}^{2} ( r ) & - cosh ( r ) sinh ( r ) \\ - cosh ( r ) sinh ( r ) & {cosh}^{2} ( r )\end{matrix} ] \\ & = [ \begin{matrix}1 & - tanh ( r ) \\ - tanh ( r ) & 1\end{matrix} ]\end{matrix} \qquad ( 135 )$$

and substitution in the definition for K yields

$$\begin{matrix}K ( r ) & = [ \begin{matrix}0 & 1 \\ 1 & 0\end{matrix} ] ( [ \begin{matrix}1 & 0 \\ 0 & 1\end{matrix} ] - [ \begin{matrix}1 & - tanh ( r ) \\ - tanh ( r ) & 1\end{matrix} ] ) \\ & = [ \begin{matrix}tanh ( r ) & 0 \\ 0 & tanh ( r )\end{matrix} ] .\end{matrix} \qquad ( 136 )$$

The results above are for 1-mode squeezed states. Generalizing to M-single-mode squeezed states is trivial since all modes are unentangled with each other. In that case, the following is obtained

$${I}_{M} - {σ}_{M - squeezed , Q}^{- 1} = [ \begin{matrix}0 & {⊕}_{j = 1}^{M} tanh ( {r}_{j} ) \\ {⊕}_{j = 1}^{M} tanh ( {r}_{j} ) & 0\end{matrix} ] \qquad ( 137 )$$

and the M-mode squeezed state kernel matrix is

$${K}_{M - squeezed} = [ \begin{matrix}{⊕}_{j = 1}^{M} tanh ( {r}_{j} ) & 0 \\ 0 & {⊕}_{j = 1}^{M} tanh ( {r}_{j} )\end{matrix} ] . \qquad ( 138 )$$

Now it is shown that the covariance matrix of the N-mode squeezed state in Eq. (131) corresponds to the following kernel matrix

$$K = c ( A ⊕ A ) = : {A}^{⊕ 2} \qquad ( 139 )$$

where the adjacency matrix A is decomposed according to Takagi's factorization as

$$A = U ( \frac{1}{c} {⊕}_{j = 1}^{M} tanh ( {r}_{j} ) ) {U}^{T} \qquad ( 140 )$$

where 1/c is a constant greater than the largest singular value of A, ensuring that every scaled singular value can be represented by the form of tanh (rⱼ). Begin by setting

$$\tilde{U} = [ \begin{matrix}U & 0 \\ 0 & {U}^{*}\end{matrix} ] \qquad ( 141 )$$

so that Eq. (131) becomes

$$σ = \tilde{U} {σ}^{′} ( r ) {\tilde{U}}^{†} \qquad ( 142 )$$

The corresponding matrix σ_{Q}=σ+I/2 can be written as

$$\begin{matrix}{σ}_{Q} & = σ + I / 2 \\ & = \tilde{U} {σ}^{′} ( r ) {\tilde{U}}^{†} + \tilde{U} I {\tilde{U}}^{†} / 2 \\ & = \tilde{U} ( {σ}^{′} ( r ) + I / 2 ) {\tilde{U}}^{†} \\ & = \tilde{U} {σ}_{Q}^{′} ( r ) {\tilde{U}}^{†}\end{matrix} \qquad ( 143 )$$

Moreover, since Ũ is unitary,

$${σ}_{Q}^{- 1} = \tilde{U} {σ}_{Q}^{′} {( r )}^{- 1} {\tilde{U}}^{†} \qquad ( 144 )$$

### Therefore,

$$\begin{matrix}I - {σ}_{Q}^{- 1} & = I - \tilde{U} {σ}_{Q}^{′} {( r )}^{- 1} {\tilde{U}}^{†} \\ & = \tilde{U} ( I - {σ}_{Q}^{′} {( r )}^{- 1} ) {\tilde{U}}^{†} \\ & = \tilde{U} [ \begin{matrix}0 & {⊕}_{j = 1}^{N} tanh ( {r}_{j} ) \\ {⊕}_{j = 1}^{N} tanh ( {r}_{j} ) & 0\end{matrix} ] {\tilde{U}}^{†} \\ & = [ \begin{matrix}U & 0 \\ 0 & {U}^{*}\end{matrix} ] [ \begin{matrix}0 & {⊕}_{j = 1}^{N} tanh ( {r}_{j} ) \\ {⊕}_{j = 1}^{N} tanh ( {r}_{j} ) & 0\end{matrix} ] [ \begin{matrix}{U}^{†} & 0 \\ 0 & {U}^{T}\end{matrix} ] \\ & = [ \begin{matrix}0 & U {⊕}_{j = 1}^{N} tanh ( {r}_{j} ) {U}^{T} \\ {U}^{*} {⊕}_{j = 1}^{N} tanh ( {r}_{j} ) {U}^{†} & 0\end{matrix} ] ,\end{matrix} \qquad ( 145 )$$

where the third equality is obtained according to Eq. (137). The kernel matrix K=X_{2M}(I_{2M}−σ_{Q}⁻¹) is then

$$\begin{matrix}K & = [ \begin{matrix}0 & {I}_{M} \\ {I}_{M} & 0\end{matrix} ] [ \begin{matrix}0 & U {⊕}_{j = 1}^{M} tanh ( {r}_{j} ) {U}^{T} \\ {U}^{*} {⊕}_{j = 1}^{M} tanh ( {r}_{j} ) {U}^{†} & 0\end{matrix} ] \\ & = [ \begin{matrix}{U}^{*} {⊕}_{j = 1}^{M} tanh ( {r}_{j} ) {U}^{†} & 0 \\ 0 & U {⊕}_{j = 1}^{M} tanh ( {r}_{j} ) {U}^{T}\end{matrix} ] \\ & = {cA}^{*} ⊕ cA \\ & = c ( A ⊕ A ) \\ & = {A}^{⊕ 2}\end{matrix} \qquad ( 146 )$$

since A is real.

### Computer Implementation

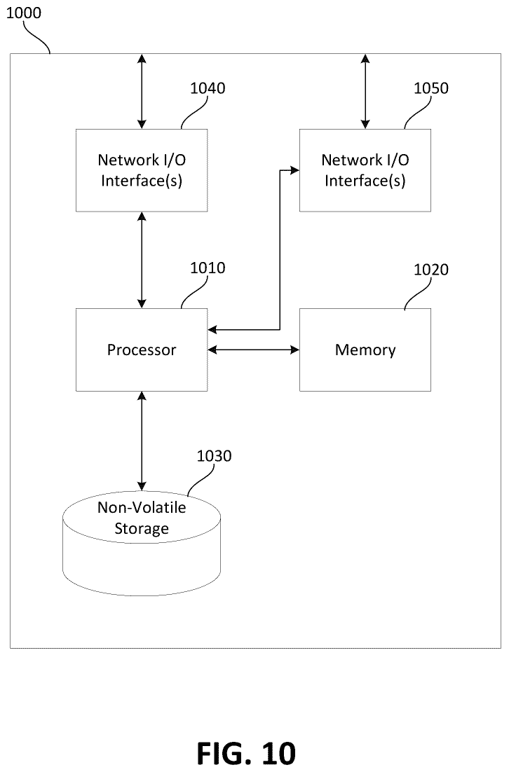

An illustrative implementation of a computer system 1000 that may be used in connection with any of the embodiments of the technology described herein (e.g., such as the method of FIG. 7 or FIG. 8) is shown in FIG. 10. The computer system 1000 includes one or more processors 1010 and one or more articles of manufacture that comprise non-transitory computer-readable storage media (e.g., memory 1020 and one or more non-volatile storage media 1030). The processor 1010 may control writing data to and reading data from the memory 1020 and the non-volatile storage device 1030 in any suitable manner, as the aspects of the technology described herein are not limited to any particular techniques for writing or reading data. To perform any of the functionality described herein, the processor 1010 may execute one or more processor-executable instructions stored in one or more non-transitory computer-readable storage media (e.g., the memory 1020), which may serve as non-transitory computer-readable storage media storing processor-executable instructions for execution by the processor 1010.

Computing system 1000 may also include a network input/output (I/O) interface 1040 via which the computing device may communicate with other computing devices (e.g., over a network), and may also include one or more user I/O interfaces 1050, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

### EQUIVALENTS

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments, and/or within ±1% of a target value in some embodiments. The terms “approximately,” “substantially,” and “about” may include the target value.

What is claimed is:

1. A method of identifying, using a compact quantum computing system, binding configurations between a first molecular structure and a second molecular structure, the method comprising:
generating a binding interaction graph describing possible binding interactions between portions of the first molecular structure and portions of the second molecular structure, the binding interaction graph being encoded as a graph adjacency matrix;
parameterizing a multimode Gaussian state vector using the graph adjacency matrix;
determining binding interactions by sampling, using holographic computing implemented on the compact quantum computing system, the multimode Gaussian state vector; and
identifying, using the determined binding interactions, cliques within the binding interaction graph, the cliques corresponding to stable binding configurations between the first and second molecular structures.

2. The method of claim 1, further comprising identifying the first molecular structure as a candidate pharmaceutical compound.

3. The method of claim 1, further comprising manufacturing the first molecular structure.

4. The method of claim 1, further comprising determining safety and/or efficacy of the first molecular structure by performing a clinical and/or nonclinical study using the first molecular structure.

5. The method of claim 1, wherein generating the binding interaction graph comprises generating a binding interaction graph describing possible binding interactions between portions of the first molecular structure and portions of the second molecular structure, the second molecular structure comprising a structure describing a biological receptor or a portion of a biological receptor.

6. The method of claim 1, wherein using holographic computing implemented on the compact quantum computing system comprises:
applying a first squeezing operation to a vacuum state of a first physical qubit to generate a first quantum state in the first physical qubit;
applying a second squeezing operation to a vacuum state of a second physical qubit to generate a second quantum state in the second physical qubit;
causing interference between the first quantum state and the second quantum state by applying a beamsplitter interaction between the first quantum state and the second quantum state; and
after applying the beamsplitter interaction:
measuring the first quantum state stored in the first physical qubit; and
initializing a third quantum state in the first physical qubit by applying a third squeezing operation to a vacuum state of the first physical qubit.

7. The method of claim 6, wherein identifying the binding interactions comprises identifying a first binding interaction by detecting a photon stored in the first physical qubit by measuring the first quantum state.

8. The method of claim 6, wherein applying the first squeezing operation comprises applying a squeezing operation having squeezing parameters determined using the graph adjacency matrix.

9. The method of claim 6, wherein applying the first squeezing operation to the first physical qubit comprises applying an electromagnetic signal to a first ancilla qubit coupled to the first physical qubit, the electromagnetic signal being configured to cause three-wave or four-wave mixing between the first quantum state and a state of the first ancilla qubit, wherein the electromagnetic signal has a frequency approximately equal to twice a resonant frequency of the first physical qubit, the first physical qubit comprising a first cavity resonator.

10-11. (canceled)

12. The method of claim 9, wherein applying the beamsplitter interaction comprises applying an electromagnetic signal to a second ancilla qubit coupled between the first and second physical qubits, the electromagnetic signal having a frequency approximately equal to a difference between resonant frequencies of the first and second physical qubits, the first and second physical qubits comprising cavity resonators.

13. (canceled)

14. The method of claim 12, wherein applying the beamsplitter interaction comprises applying the electromagnetic signal to the second ancilla qubit, the electromagnetic signal configured to cause four-wave mixing between the first quantum state and the second quantum state.

15. The method of claim 6, further comprising:
applying a third squeezing operation to a vacuum state of a third physical qubit to generate a fourth quantum state; and
after applying a beamsplitter interaction between the first quantum state and the second quantum state and before measuring the first quantum state:
first, applying a beamsplitter interaction between the second quantum state and the fourth quantum state; and
second, applying a beamsplitter interaction between the first quantum state and the second quantum state.

16. A system, comprising:
a compact quantum computing system;
at least one computer hardware processor; and
at least one non-transitory computer readable medium storing processor-executable instructions that, when executed by the at least one computer hardware processor, cause the at least one computer hardware processor to perform a method of identifying binding configurations between a first molecular structure and a second molecular structure, the method comprising:
generating a binding interaction graph describing possible binding interactions between portions of the first molecular structure and portions of the second molecular structure, the binding interaction graph being encoded as a graph adjacency matrix;
parameterizing a multimode Gaussian state vector using the graph adjacency matrix;
determining binding interactions by sampling, using holographic computing implemented on the compact quantum computing system, the multimode Gaussian state vector; and
identifying, using the determined binding interactions, cliques within the binding interaction graph, the cliques corresponding to stable binding configurations between the first and second molecular structures.

17. The system of claim 16, wherein the method further comprises identifying the first molecular structure as a candidate pharmaceutical compound.

18. The system of claim 16, wherein generating the binding interaction graph comprises generating a binding interaction graph describing possible binding interactions between portions of the first molecular structure and portions of the second molecular structure, the second molecular structure comprising a structure describing a biological receptor or a portion of a biological receptor.

19. The system of claim 16, wherein using holographic computing implemented on the compact quantum computing system comprises causing a controller to:
apply a first squeezing operation to a vacuum state of a first physical qubit to generate a first quantum state in the first physical qubit;
apply a second squeezing operation to a vacuum state of a second physical qubit to generate a second quantum state in the second physical qubit;
cause interference between the first quantum state and the second quantum state by applying a beamsplitter interaction between the first quantum state and the second quantum state; and
after applying the beamsplitter interaction:
measure the first quantum state stored in the first physical qubit; and
initialize a third quantum state in the first physical qubit by applying a third squeezing operation to a vacuum state of the first physical qubit.

20. The system of claim 19, wherein identifying the binding interactions comprises identifying a first binding interaction by detecting a photon stored in the first physical qubit by measuring the first quantum state.

21. The system of claim 19, wherein applying the first squeezing operation comprises applying a squeezing operation having squeezing parameters determined using the graph adjacency matrix.

22. The system of claim 19, wherein applying the first squeezing operation to the first physical qubit comprises applying an electromagnetic signal to a first ancilla qubit coupled to the first physical qubit, the electromagnetic signal being configured to cause three-wave or four-wave mixing between the first quantum state and a state of the first ancilla qubit, wherein the electromagnetic signal has a frequency approximately equal to twice a resonant frequency of the first physical qubit, the first physical qubit comprising a first cavity resonator.

23. (canceled)

24. The system of claim 19, wherein applying the beamsplitter interaction further comprises:
determining a covariance matrix using the graph adjacency matrix; and
applying a beamsplitter interaction having beamsplitting parameters based on the covariance matrix.

25. The system of claim 24, wherein applying the beamsplitter interaction comprises applying an electromagnetic signal to a second ancilla qubit coupled between the first and second physical qubits, the electromagnetic signal having a frequency approximately equal to a difference between resonant frequencies of the first and second physical qubits, the first and second physical qubits comprising cavity resonators.

26. The system of claim 25, wherein applying the beamsplitter interaction comprises applying the electromagnetic signal to a transmon qubit.

27. The system of claim 25, wherein applying the beamsplitter interaction comprises applying the electromagnetic signal to the second ancilla qubit, the electromagnetic signal configured to cause four-wave mixing between the first quantum state and the second quantum state.

28. The system of claim 19, the method further comprising causing the controller to:
apply a third squeezing operation to a vacuum state of a third physical qubit to generate a fourth quantum state; and
after applying the beamsplitter interaction between the first quantum state and the second quantum state and before measuring the first quantum state:
first, apply a beamsplitter interaction between the second quantum state and the fourth quantum state; and
second, apply a beamsplitter interaction between the first quantum state and the second quantum state.

29-42. (canceled)
